(19) United States
(12) Patent Application Publication
(10) Pub. No.: US 2007/0275080 A1
(43) Pub. Date: Nov. 29, 2007

(54) Polymer-Based Microstructures

(75) Inventors: Bryan E. Laulicht, Roslyn Heights, NY (US); Sasha Bakhru, Loudonville, NY (US)

Correspondence Address: DARBY & DARBY P.C., P.O. BOX 770, Church Street Station, New York, NY, 10008-0770, US

(73) Assignee: ENGINEERED RELEASE SYSTEMS INC., RENSSELAER, NY (US)

(21) Appl. No.: 10/577,785

(22) PCT Filed: Oct. 29, 2004
(86) PCT No.: PCT/US04/36158

§ 371 Date: Jul. 5, 2007

Related U.S. Application Data
(60) Provisional application No. 60/516,224, filed on Oct. 31, 2003.

(51) Int. Cl. A61K 9/14; B41J 2/01

(52) U.S. Cl. 424/493; 347/20; 424/497

(57) ABSTRACT

The present invention relates to the fields of controlled release of drugs, proteins, nucleic acids, and other pharmaceuticals. It also relates to delivery systems for these agents and other compounds. The invention also relates to stable encapsulation of cells and molecules. The invention provides a population of microstructures comprising a permeable polymer shell, wherein the standard variance in the volume of the microstructures is usually less than or equal to 20%, preferably 10%, of the mean, and wherein the diffusion characteristics of the polymer shell vary within the population of microstructures. It also provides for an apparatus and a method of forming a population of microstructures, which method for making microstructures by introducing drops of a polymer solution into a receiving solution under conditions that permit cross-linking of the polymer in the receiving solution. Microstructures of calcium-cross-linked alginate with a chitosin capsule are disclosed.

### RELATED APPLICATIONS

The present application claims priority to U.S. Provisional Application Ser. No. 60/516,224 filed Oct. 31, 2003 of which the instant application claims priority pursuant to 35 U.S.C. §119(e), and which is specifically incorporated herein by reference in its entirety.

### FIELD OF THE INVENTION

The present invention relates to the fields of controlled release of drugs, proteins, nucleic acids, and other pharmaceuticals. It also relates to delivery systems for these agents and other compounds. The invention also relates to stable encapsulation of cells and molecules.

### SUMMARY OF THE INVENTION

The present invention provides for a population of microstructures having a volume of less than or equal to about 10 nL comprising of a cross-linked polymer, wherein the standard variance in the volume for the microstructures is less than or equal to 20%, preferably 10%, of the mean. The invention also provides for microstructures comprising a permeable polymer shell, wherein the variance in the volume is less than or equal to 10% of the mean.

In one embodiment of this invention, microstructures are loaded with active agents. Active agents may be front-loaded or back-loaded depending upon the size of the active and the molecular mass cut-off of the polymer blend of the microstructures. In an alternative embodiment, the core of the microstructures comprises a single cell. The diffusion characteristics of the polymer shell or the time for maximum release for molecules contained in the microstructures can vary within the population of microstructures. By continuously varying the diffusion characteristics of the polymer shells from microstructure to microstructure, time-dependent delivery, in some instances matching closely the natural cycles of certain human-derived biological macromolecules in individuals, can be obtained.

A manufacturing method of the present invention comprises introducing drops of a polymer solution into a receiving solution under conditions that permit cross-linking of the polymer in the receiving solution, wherein the standard variance of the droplets is less than or equal to 20%, preferably 10%, of the mean. The cross-linked polymer droplet can then be further coated through interaction with a polymer bath, resulting in a permeable polymer shell. The invention also provides for a drop-forming apparatus comprising a plurality of orifices of uniform size spaced far enough apart so that drops ejected from the orifices do not combine, a reservoir in fluid communication with the plurality of orifices, and an activation means for ejecting drops from each orifice.

### DESCRIPTION OF THE DRAWINGS

FIG. 1. A depiction of the formation of spherical templates through the introduction of polymer droplets into a receiving solution comprising a cross-linking agent. Once the droplets are received, the cross-linking agent diffuses into the droplet, resulting in cross-linking of the polymer molecules and formation of a semi-solid template.

FIG. 2. A depiction of the formation of microspheres or coated spherical templates through the interaction of templates with a polymer bath. Once the templates are received, the polymer diffuses into the template forming an outer shell. The residence time of the templates in the polymer bath and the composition of the polymer bath affect the thickness of the outer shell formed within the template. The volume of each microstructure does not appreciably change once the templates have been coated within or infused with a polymer.

FIG. 3. A perspective view of a specific embodiment of a modified inkjet cartridge.

FIG. 4. A cutaway perspective view of the modified inkjet cartridge of FIG. 3, illustrating the plurality of nozzles, a plurality of activation elements, a plurality of electrical contacts, and a plurality of electrical conductors.

FIG. 5. A schematic diagram of the specific embodiment the amplification circuit.

FIG. 6. A photomicrograph (100× magnification) of the spherical droplets resulting from a specific embodiment of the modified inkjet cartridge.

FIG. 7. A cutaway perspective view of a practical design for a potential drop-forming apparatus.

FIG. 8. A schematic diagram of a practical design for a potential drop-forming apparatus FIG. 7, illustrating the plurality of modified inkjet cartridges and the controller.

FIG. 9. A depiction of the formation of microcapsules through the introduction of droplets comprising of a cross-linking agent, i.e. calcium cation, into a polymer solution. Once the droplets are received, the cross-linking agent diffuses to the interface of the droplet were the polymer cross-links onto the surface of the droplet to form a membrane therearound. The volume of the microcapsules is larger than the volume of the droplets.

FIG. 10. A depiction of the formation of a polymer-blended microcapsule through the interaction of microcapsules with a polymer bath. The polymer diffuses into the shell of the microcapsule. The residence time of the microcapsules in the polymer bath does not affect the thickness of the shell of the microcapsule.

FIG. 11. A schematic diagram of a spherical template.

FIG. 12. A schematic diagram of the release profile of a spherical template.

FIG. 13. A schematic diagram of a population of spherical templates comprising of the same volume.

FIG. 14. A schematic diagram of the release profile of population of spherical templates comprising of the same volume.

FIG. 15. A schematic diagram of a unilamellar microstructure.

FIG. 16. A schematic diagram of the release profile of a unilamellar microstructure.

FIG. 17. A schematic diagram of two unilamellar microstructures with varying shell thicknesses.

FIG. 18. A schematic diagram of the release profile of a mixture comprising unilamellar microstructures with varying shell thicknesses.

FIG. 19. A schematic diagram of chitosan capsule wall thickness.

FIG. 20. A schematic diagram of the release profile of a chitosan capsule containing analyte in both the outer wall and core of the capsule.

FIG. 21. A schematic diagram of the Vitamin B-12 release profile from an alginate capsule of uniform wall thickness.

FIG. 22. A photomicrograph (200× magnification) of alginate microcapsules in 0.25 w/v % CaCl₂ solution.

FIG. 23. A photomicrograph (200× magnification) of alginate microcapsules in distilled water.

FIG. 24. A photomicrograph (200× magnification) of alginate microcapsules in distilled water after two hours.

FIG. 25. A schematic diagram of an inkjet cartridge setup containing a cell suspension positioned over a receiving bath.

### DETAILED DESCRIPTION

The present invention provides a technology for the formation of polymer-based microstructures whose shape, volume, and diffusion rate of encapsulated materials can be accurately controlled. By microstructure is meant micron-sized, i.e., on the order of 0.1 micron to 100 micron scale, preferably in the 1-10 or 10-50 micron scales, solid or semi-solid structure that can be multilayered, having at least one outer shell, and in some cases an inner cross-linked core.

The present invention for the first time provides a controlled release system capable of reproducibly generating any release profile defined by a summation of sigmoidal release curves. Thus, without employing mechanical devices, and avoiding multiple injections timed for a particular time of day, one can achieve any desired release pattern, e.g., release timed to diurnal or circadian rhythms, meals, activity, or any other schedule. This achievement represents a significant advance in the field of controlled release drug delivery.

The present invention involves a confluence of three distinct achievements. The first is the development of a microstructure encapsulation or shell-formation methodology that results in a population of microstructures with a continuous or discontinuous variation in wall thickness, providing a range of times to maximum release for molecules contained in the microstructures. Second is the development of technology to produce a population of microstructures of uniform size, e.g., the standard variance in the volume of microstructures is less than 20% of the mean for polymer solutions with outlying fluid properties such as extremely high or low viscosity, surface tension, etc., or less than 10% of the mean in standard formulations, preferably less than about 5% of the mean, and in some instances within 2% of the mean. This reproducibility is achieved even at very small volumes, e.g., in microstructures having volumes of 10 uL or less, 5 mL or less, 2 mL or less, and even about 10 pL. Such microstructures can be less than or equal to 100 microns along the longest axial dimension. For example, particles can have a size of from 0.1 to 100 microns along the longest axis, e.g., 1 to about 50 microns or about 5 to about 20 microns. It should be noted that the populations can vary in size, but the invention provides for microstructures in any given population of a well-defined size, e.g., 30±1.5 microns.

In addition, as described below, in certain embodiments the invention also provides for generation of regular spherical microstructures. An advantage of spherical microstructures is the uniform diffusion rates of material in or out of the sphere. Irregular structures will have irregular microenvironments at various points, resulting in different diffusion rates and less control over diffusion on the macroscopic scale.

Finally, the present invention permits generation of highly reproducible microstructure populations from batch to batch, which makes them desirable for pharmaceutical preparations.

The microstructures of the invention can deliver all manner of active agents, as discussed below. Moreover, they can encapsulate individual cells, which in turn can divide a number of times to form daughter cells and cell clones. Encapsulated cells can exchange nutrients and metabolites through the shell of the microstructure, but are protected from external immune recognition.

In the present invention, the polymer templates can be formed through interaction of polymer droplets with a receiving solution comprising a primary solvent, which induces cross-linking of the polymer droplets. In preferred embodiments, the droplets have a reproducible volume. Presence of a secondary solvent in a receiving solution that increases the interfacial tension between the droplets and the receiving solution results in the droplets adopting a structure that minimizes contact with the receiving solution, i.e., a spherical shape, as the cross-linking proceeds. In specific embodiments, the receiving solution is immiscible with the polymer solution due to either the transient or permanent immiscibility of the receiving solution with the polymer solution.

An outer polymer shell or coating within the template may be formed through interaction of the templates with a polymer bath. The addition of the outer polymer shell does not alter the volume of the templates; i.e., the polymer shell grows within the templates. With further processing, a microcapsule can be formed by dissolving the cross-linking of the polymer that forms the template core of the microstructure. As used herein, a “microcapsule” refers to a microstructure with a non-solid or semi-solid core.

Alternatively, microcapsules can be formed through the introduction of droplets comprising a cross-linking agent into a polymer-receiving solution. In this embodiment reproducible size of the droplets is desirable. Again, the use of a secondary solvent in a receiving solution that increases the interfacial tension between the droplets and the receiving solution results in the droplets adopting a structure that minimizes contact with the receiving solution, i.e., a sphere. In this embodiment, once the droplets are introduced into the polymer-receiving solution, the cross-linking agent diffuses to the interface of the droplets where the polymer cross-links onto the surface of the droplet to form a shell or coating. The volume of the microcapsules prepared this way is greater than the volume of the droplets, i.e., the polymer shell grows on the outside of the droplets, which will vary in size depending on the cross-linker concentration. A polymer-blended microcapsule may be formed through interaction of the microcapsules with a polymer bath.

In a specific embodiment of the invention, microstructures are loaded with active agents, and serve as controlled release carriers. The loaded polymer-based microstructures can be reacted with a targeting agent to enable site-specific delivery of the active agent.

The factors that govern the release rate of the loaded structures, i.e. thickness of the outer polymer layer and the volume and shape of the templates, can be accurately controlled, as discussed below. By continuously varying the shell thicknesses of the microstructure to microstructure, time-dependent delivery, in some instances matching closely the natural cycles of certain human-derived biological macromolecules in individuals, can be obtained.

Controlled release pharmaceutical preparations regulate the release of the incorporated active agent(s) over time and comprise preparations with a delayed, a sustained, a controlled, or an extended release, so they accomplish therapeutic or convenience objectives not offered by conventional dosage forms. Controlled release of active agent(s) allows the medical provider to simplify the patient's posological scheme by reducing the amount of recommended daily intakes of a drug, and this in turn improves patient's compliance.

While a number of drugs have been formulated in a controlled released mode, the easy modulation of the pharmacokinetic/pharmacodynamic profile of “large” active agent(s), i.e., macromolecules such as proteins, peptide hormones, and nucleic acids, in controlled release method has not been achieved. By “large” active agents is meant drugs whose molecular weight up to about 200 kDa in diffusion-limited formulations and up to 2 MDa in the environmentally cued formulations, preferably drugs whose molecular weight is on the range of 1 to 150 kDa for diffusion-limited formulations. The invention meets this need for effective drug release of large molecules.

In alternative embodiments, the core of a microcapsule comprises a single cell. This is known in the art as single-cell encapsulation. Single-cell encapsulation can be achieved utilizing either of the above discussed methods for microcapsule formation.

For example, reproducible volume droplets of a polymer-cell suspension can be introduced into a receiving solution that cross-links the droplets, such that each droplet contains, on average one cell. Again, use of a secondary solvent in receiving solution that increases the interfacial tension between the droplets and the receiving solution, results in the droplets adopting a structure that minimizes contact with the receiving solution, i.e., a sphere. However, to protect cell viability, particularly of eukaryotic cells, it may be necessary to use isotonic, buffered aqueous solutions for the polymer (or cross-linker) containing the cells and for the receiving solution. An outer polymer shell can be formed through interaction of the templates with a polymer bath, just as with the microstructures described above. The templates can be dissolved by the introduction of an appropriate solvent, resulting in encapsulated cells. The microcapsules comprising live cells can be separated from those which contain dead cells or no cells. The encapsulated cells can then be coated to enable specific targeting, as discussed above for the microstructures.

Alternatively, single-cell encapsulation can be achieved without the use of a template. For example, reproducible volume droplets of a suspension comprising cells and a cross-linking agent are introduced into a polymer-receiving suspension, such that each droplet contains, on average one cell. Once the droplets are received into the polymer-receiving solution the cross-linking agents diffuses to the interface of the droplet and the polymer cross-links on surface of the droplet. Again, use of a secondary solvent in receiving solution that increases the interfacial tension between the droplets and the polymer-cell receiving suspension, results in the droplets adopting a structure that minimizes contact with the receiving solution, i.e., a sphere. The interaction of microcapsules whose liquid cores contain cells with a polymer bath results in a polymer-blended encapsulate cells. Like before, the microcapsules comprising live cells can be separated from those comprising dead cells or no cells.

Cell encapsulation is a promising therapy for a variety of diseases such as diabetes, severe liver failure, and other disorders caused by specific deficiencies (Canaple et al., J. Biomater. Sci. Polymer Edn. 2002, 13:783-796). The capsule in which cells are entrapped is a select permeable membrane that affords the cell protection from an attack by the host immune system. However, the encapsulating medium also serves as a barrier to receiving metabolites and excreting waste products. Consequently, the greatest surface area to volume ratio per encapsulated cell is desired and it is therefore advantageous to be able to encapsulate single cells (Canaple et al., supra). While multiple cells have been encapsulated, to the best of our knowledge, there are no published results of single-cell encapsulation of eukaryotic cells. In this specific embodiment the needs for single-cell encapsulation are met.

The term “population” is used in this application to mean a collection or group of microstructures. The population can result from a single batch process or from a combination of groups from different batch processes.

As mentioned above, a “microstructure” is a micron-scale particle of a polymer shell embedded in a cross-linked polymer. The cross-linked polymer may be present in the center “core” of the microstructure, or the core may be free of gelled polymer. Microstructures can be of regular or irregular shape, including spheroids, ellipsoids, and tear drops.

The term “template” refers to microsized semi-solid or gelatinous cross-lined polymer-based structure that can serve as the core in a multilayered microstructure. The template can have properties of a hydrogel. If the template is part of the microstructure, the polymer and cross-linker must be compatible with any active agent to be loaded. Acceptable template polymers include alginate, collagen and collagen derivatives, cellulose and cellulose derivatives, agarose, and sepharose.

The term “cross-linked” in any of its grammatical forms, used in conjunction with a polymer to form a template of the invention, refers to any covalent or electrostatic linkage of the polymers that from the template to form a network of polymers. This network results in greater viscosity, to the point of a semi-solid or gelatinous phase. Exemplary cross-linking agents include calcium (and other multivalent metal cations), which cross-link alginate and other anionic polymers via electrostatic interactions, and chemical cross-linkers, including photoactivated cross-linkers, which covalently join the polymers.

The term “standard variance” refers to the variance of the population within two standard deviations from the mean.

An “axial distance” is the distance from one side of a particle through the geometric center to another side. The axial distance of a sphere is its diameter.

The term “shell” or “coating” refers to a complex of a polymer infused into the template matrix. Exemplary polymers include chitosan and other cationic cellulose derivatives when the template is alginate or another anionic polymer. The shell creates a more stable, solid structure that is semi-permeable to molecules below the molecular mass cut off.

The term “polymer” as used herein refers to a molecule containing a plurality of covalently attached monomer units. A polymer for use in a template can be cross-linked. The term polymer also includes branched, dendrimeric, linear, and star polymers as well as both homopolymers and copolymers.

As used herein, the term “organic solvent” is intended to mean any carbon-based liquid solvent, preferably one that is non-polar, and more preferably one that is immiscible in water. Exemplary organic solvents include the hydrocarbons that are liquids at room temperature, including hexane, heptane, octane, nonane, decane, and mixtures thereof; petroleum ether; mineral oil, olive oil, and mixtures thereof.

The term “active agent” refers to any chemical compound that is loaded into the microstructure or microcapsule. As used herein, the term “active agent” refers to one or more compounds. Active agents include, but are not limited to, drugs, proteins, nucleic acids, flavorants, nutrients, hormones, and small molecules. The terms also encompass pharmaceutically acceptable, pharmacologically active derivatives of those active agents specifically mentioned herein, including, but not limited to, salts, esters, amides, active metabolties, isomers, analogs, and the like.

The phrase “pharmaceutically acceptable” refers to molecular entities, at particular concentrations, and compositions that are physiologically tolerable and do not typically produce an allergic or similar untoward reaction, such as gastric upset, fever, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in humans.

A “formulation” refers to a medium for the preservation or administration, or both, of loaded microstructures.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “front-loadable” refers to incorporating an active into the polymer solution of a drop forming apparatus. This is typically done when the active exceeds the molecular mass cut-off of the polymer blend of the microcapsules.

The term “back-loadable” refers to incorporating an active into the receiving solution of a drop forming apparatus. This is typically done when an active is below the molecular mass cut-off of the polymer blend of the microcapsules.

The various aspects of the invention will be set forth in greater detail in the following sections. This organization into various sections is intended to facilitate understanding the invention, and is no way intended to be limiting thereof.

Microstructures

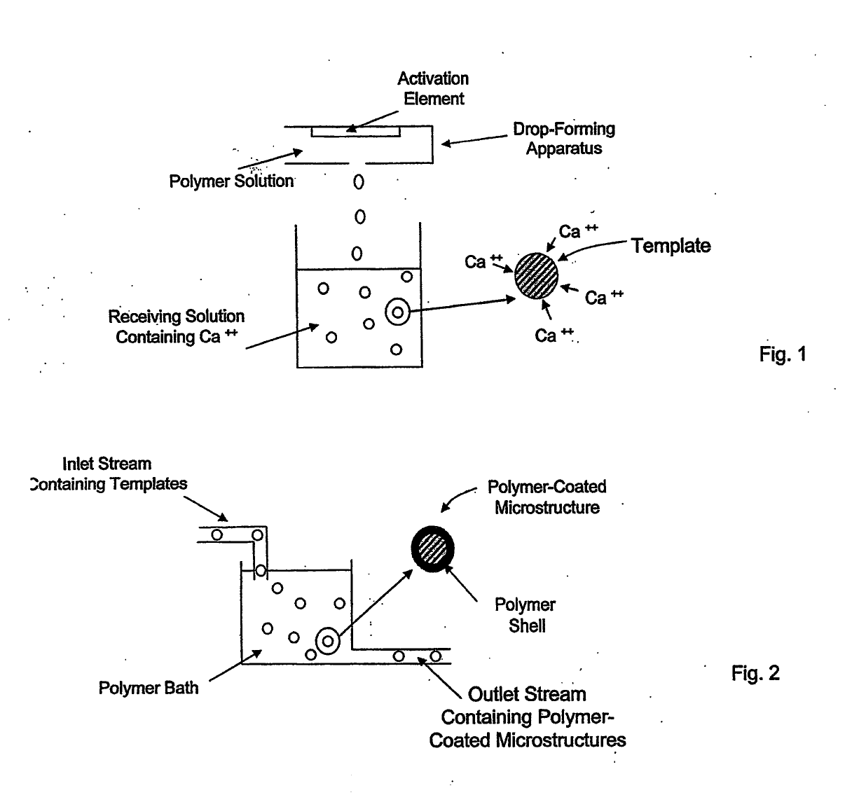

In the present invention, microstructures can be multilayered, including a template and at least one outer shell within the template. Polymer templates can be formed through interaction of polymer droplets of reproducible volume with a receiving solution, which induces cross-linking of the polymer droplets. FIG. 1 depicts the formation of templates by the introduction of polymer droplets into a receiving solution containing a cross-linking agent, e.g., drugs of an alginate solution into a solution containing a multivalent cation. The cation diffuses into the droplet, resulting in electrostatic cross-linking of alginate and the formation of a template. As discussed above, an outer polymer shell within the template may be formed through interaction of the templates with a polymer bath. The addition of the outer polymer shell does not alter the volume of the templates, i.e., the polymer shell grows within the templates. FIG. 2 depicts the formation of a microstructure having a template and one outer shell. Additional shells or coatings can be added if desired.

Liquid-core microstructures or microcapsules can be formed by solubilizing the template polymers. Alternatively, microcapsule structures result from forming template capsules by introducing drops of a cross-linking agent into a receiving solution containing a cross-linkable polymer, e.g., a calcium solution into an alginate solution. These template capsules become more stable, rigid, and less permeable by infusing them with a shell or coating polymer, such as chitosan.

Template Manufacturing

The manufacturing of microstructures involves the production of templates. Template manufacturing employs the reproducible formation of microsized polymer droplets and the interaction of these droplets with a receiving solution, the latter resulting in cross-linking the polymer droplet. “Reproducibility” is defined as varying in volume by no more than 10%, preferably by no more than 2%. Furthermore, template manufacturing may include the use of solvent exchange to selectively replace the solvent(s) of the receiving solution after the core has formed.

Drop-Forming Technology

Template production employs a drop-forming apparatus that is capable of reproducible single-droplet generation. A drop-forming apparatus comprises at least one nozzle or orifice, preferably a plurality or nozzles or orifices, a supply reservoir, an activation element, and a controller. The nozzle or orifice is in fluid communication with the supply reservoir. The diameter of the nozzles affect the volume of the droplets formed. In general, the diameter of the spherical droplet ejected is roughly equal to the diameter of the nozzle. The activation element causes the ejection of droplets out of the nozzles or orifices. The controller controls the activation of the activation element. Drop-forming apparatuses include, but are not limited to, modified inkjet cartridges and capillary tubes.

Many factors affect the reproducibility, the number, and the volume of droplets. These include, but are not limited to, the spacing and size of the nozzles, the activation element, the controller, and the distance between the nozzles and the receiving solution. Ambient temperature and humidity also affect reproducibility. Modifications to ensure the reproducible formation of droplets can include, but are not limited to, the factors set-forth above.

Inkjet technology. The material comprising nozzles or inkjets can include, but is not limited to, gold foil and silica wafers. Commercially available inkjet cartridges can have nozzles diameters that range from a fraction of a micron to hundreds of microns. The ratio of distance between nozzles to the size of the nozzle may affect droplet reproducibility. For example, in adapting a commercially available inkjet, the inkjet cartridge is modified to augment this ratio to prevent droplet coalescence; the inkjet cartridge is modified so that a select number of activation elements, corresponding to nozzles located at sufficient distances from one another, are utilized. This ensures that the inter-nozzle spacing is large enough to avoid drop combination. Generally, the distance between nozzles or orifices will be at least an order of magnitude greater than the nozzle or orifice diameter.

Activation element. The activation element causes the ejection of droplets from the nozzles. A variety of activation elements can be used in inkjet technology. A nonexclusive list includes resistors, piezoelectric solids, and air pulses.

Using heat technology to activate the formation and ejection of droplets is known in the art as bubble jet printing. In this technique, tiny resistors create heat which vaporizes the liquid to create a bubble. As the bubble expands, some of the liquid is pushed out of a nozzle into the receiving solution. When the bubble cavitates, a vacuum is created pulling more liquid into the nozzle from the supply reservoir.

Alternatively, piezoelectric crystals can be utilized as a means for the formation and ejection of droplets. In this technique, a piezoelectric crystal is located at the back of each nozzle. The crystal receives a tiny electric charge that causes a dimensional response. When the crystal expands in the desired dimension, it forces a tiny amount of liquid out of the nozzle. When it vibrates out, it pulls some more liquid into the supply reservoir to replace the ejected liquid.

In yet another alternative, air pulses can be used as a means for the formation and ejection of droplets. In specific embodiments, air pulses are introduced near the end of the nozzle. As the air pulse is introduced, some of the liquid is pushed out of the nozzle. Once the air bubble rises above the nozzle into the main supply reservoir, a vacuum is created pulling more liquid into the nozzle from the supply reservoir. In another embodiment of this technique, the supply reservoir is under vacuum. The introduction of atmospheric pressure causes liquid to leak from the nozzles.

Controller. The controller may include, but need not be limited to, a constant pulse generator providing pulses at a constant frequency. For example, the controller can be a serial, USB, etc., port on a computer providing a pulse train determined by software. The controller determines the amplitude, duration (width), and frequency of the pulses that activate the activation element. The pulse amplitude, width, and frequency affect the rate of droplet formation, the volume of the droplets formed, and the reproducibility of single-droplet generation. Multiple combinations of pulse width, pulse amplitude, and frequency can enable reproducible single-droplet generation for a particular polymer solution. In general, as the viscosity of the polymer solution increases, so too does the amplitude and/or the duration of the pulses required to reproducibly form single-droplets. If the pulse is too strong (pulse width too wide and/or pulse amplitude too high), the activation element may not have time to recover before it is pulsed again and multiple droplets can form from a single pulse. Alternatively, if the activation element receives too weak a pulse (too short in duration or too low in amplitude), the surface energy of the polymer solution may be too great for activation element to overcome, and no fluid can be ejected. In the latter case an amplification circuit can be utilized to enable reproducible formation of microsized droplets.

Distance between the nozzle plate and the receiving solution. The distance the droplet must travel before reaching the receiving solution will affect the shape of the droplet. If the droplet height is too great, the droplet may form a teardrop tail region or evaporate before reaching the receiving solution. If the droplet height is too small, the droplet may fail to penetrate the surface of the receiving fluid and pancake.

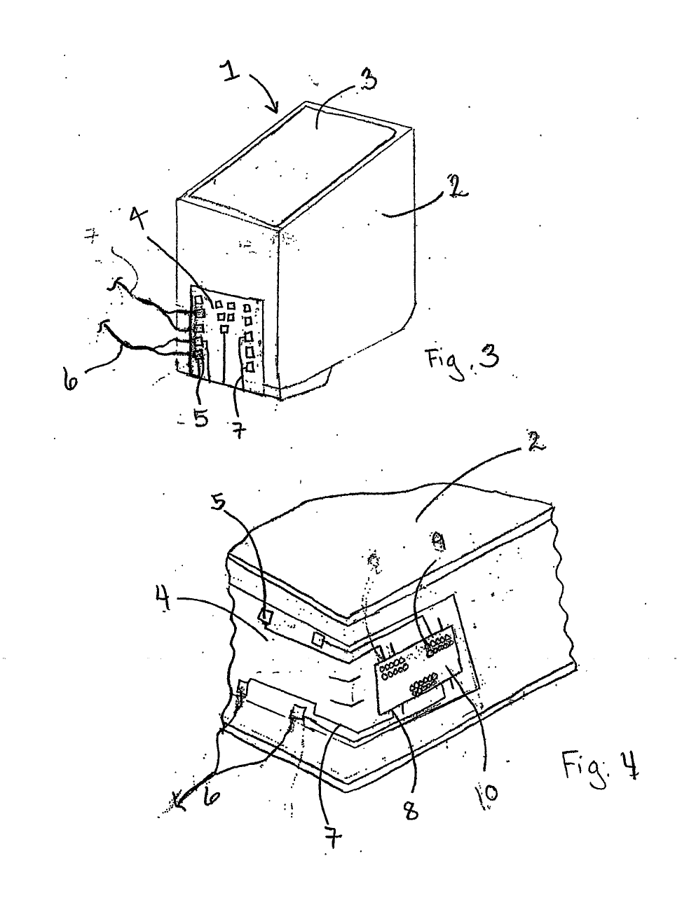

In a specific embodiment of this invention, a plurality of modified commercially available inkjet cartridges, filled with a polymer solution, are utilized to reproducibly form droplets. FIGS. 3 and 4 are schematic diagrams of a modified inkjet cartridge. The modified inkjet cartridge 1 comprises a hard case covering 2, a supply reservoir 3 disposed within, a flexible conductor 4 disposed on surface, a plurality of electrical contacts 5, a pair of soldered wire leads 6, a plurality of electrical conductors 7, a plurality of activation elements 8, a plurality of nozzles 9, and a jet plate assembly 10. A supply reservoir 2 is in fluid communication with a plurality of nozzles 9. The flexible conductor 4 comprises a plurality of electrical conductors 7, wherein the assembly side of each of the plurality of conductors 7 is connected to the jet plate assembly 10 and wherein the contact side is connected to an electrical contact 5. The jet plate assembly 10 and is disposed on the surface of the flexible conductor 4. The jet plate assembly 10 comprises a plurality of activation elements 8 and a plurality of ink channels (not shown). In addition, the jet plate assembly 10 is associated with a plurality of nozzles 9. The wire leads 6 are soldered to a pair of electrical contacts 5. The activation element 8 is treated as a resistor. Each of the plurality of nozzles 9 is located proximate to its associated nozzle to enable the direct heating of the polymer solution delivered by its associated channel. The soldered wire leads 6 receive a pulse, the pulse is conducted through the electrical conductors 7, and is received by the activation elements 8, which cause the formation and ejection of droplets from the nozzles 9.

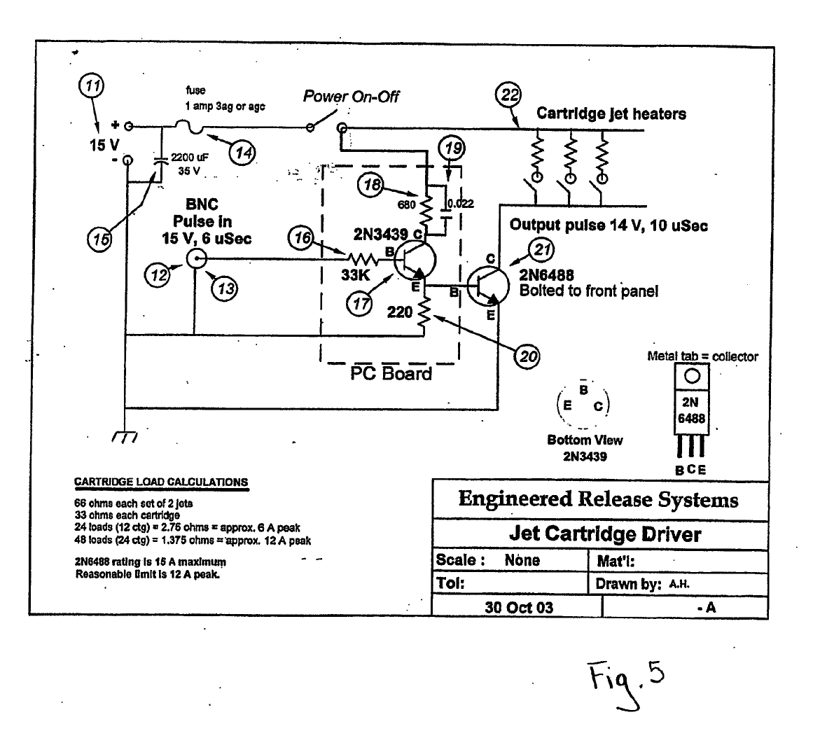

This embodiment uses a pulse generator as the controller, and employs an amplification circuit. FIG. 5 is a schematic diagram of an amplification circuit, which employs a modified Darlington configuration. The amplification circuit can be a 15V constant power supply 11, a pulse generator 12, a ground 13, a fuse 14 which is in series with the 15V constant power supply 11, a first capacitor 15 which is in parallel with a 15V constant power supply 11, a first transistor 16, a 33,000 ohm resistor 17 which is connected to the base of the first transistor 16, a second capacitor 18, a 680 ohm resistor 19 which is in parallel with the second capacitor 18, a 220 ohm resister 20 which is connected to the emitter of the first transistor 17, a second transistor 21, an input and an output lead 22. The combination of the 680 ohm resistor 19 in parallel with the second capacitor is connected to the collector of the first transistor 16. The base of the second transistor 21 is connected to the emitter of the first transistor 16. The wire leads 6 of the modified inkjet cartridge are wired to the input and output leads 22. The pulse generator 12 supplies an input pulse that drops over 33,000 ohm resistor 17. This supplies current to the base of the first transistor 16. The first transistor 16 amplifies the current and provides the base current to the second transistor 21. The second transistor 21 allows approximately six amps to pass, which dissipates in the activation elements (resistors) 8 of the modified inkjet cartridges 1.

In order to facilitate reproducible single droplet generation, a partial number of activation elements in a modified inkjet cartridge can be wired to the controller and a combination of pulse frequency, amplitude, and width can be determined. FIG. 6 depicts spherical droplets 23 resulting from this embodiment.

Cartridge pressurization. Surfactant concentration, ambient conditions, and solution density can be modified to create the necessary equilibrium that allows expulsion of media while causing retention at the pores of a drop generating device. In a specific embodiment of this invention, dynamic equilibrium is used to mechanically achieve the necessary equilibrium. Pressure is one control of dynamic equilibrium.

In one embodiment, pressurization can be achieved by a linearly actuated piston/cylinder. In a specific embodiment, a syringe with a silicone gasket affixed to a chamber made of polycarbonate serves as the liner actuated piston/cylinder. The silicone gasket is clamped between the polycarbonate chamber and the fluid reservoir of the capsule generator. The volume of the fluid in the reservoir and the volume of the piston/cylinder define the initial volume (Volₒ). By displacing the syringe relative to the syringe cylinder, the total volume of fluid reservoir can be controlled, as explained by Boyle's law. Varying the total volume of the fluid reservoir directly translates to controlling the pressure within the reservoir. Pressurization allows for many more polymer blends to be used as front-loadable and back-loadable actives in a drop generating device.

### Alternative Drop-Forming Technology

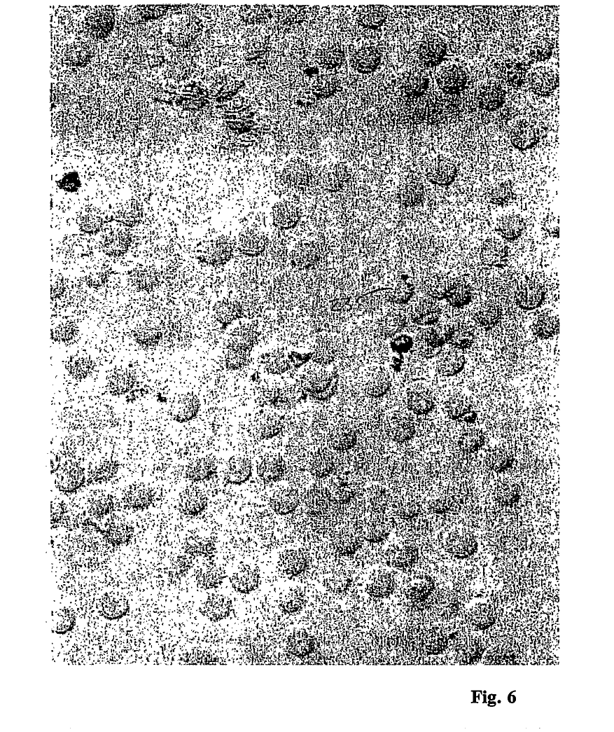

An alternative apparatus for the reproducible formation of droplets is shown in FIG. 7. A inkjet cartridge 24 has a supply reservoir 25 in fluid communication with a plurality of nozzles 27. The distance between nozzles 28 is at least one order of magnitude greater than the diameter of the nozzles. The piezoelectric activation element 26 causes the formation and ejection of droplets from the nozzles 27. In FIG. 8, a plurality of inkjet cartridges 24 is shown connected to a controller 29. Other devices that may achieve reproducible single droplet generation include, but is not limited to, capillary tubes.

### Polymer & Receiving Solutions

The interaction of microsized droplets of a polymer solution with a receiving solution results in the formation of templates or cross-linked polymer droplets. The use of a system of a primary solvent that contains the cross-linking agent and a secondary solvent that increases the interfacial tension between the droplets and the receiving solution, results in the droplets adopting a structure that minimizes contact with the receiving solution, i.e., a sphere. In specific embodiments, the primary or secondary solvent of the receiving solution is at least transiently immiscible with the polymer solution.

The polymer droplet must cross-link rapidly to prevent deformation due to collisions but not so rapidly that the droplets do not have sufficient time to recover from their deformation upon entry into the receiving solution. Additional factors that affect the shape and volume of templates include, but are not limited to, the speed at which the receiving solution is stirred (if at all) and the difference in hydrophobicity between the polymer and receiving solutions. For example, an aqueous polymer solution can be introduced into an non-polar organize receiving solution, e.g., an organic solvent as described above.

The cross-linking process of the polymer droplets may be a physical or a chemical phenomenon. Additionally, the polymer and receiving solutions may be classified as single-component or dual-component cross-linked polymer systems. Single-component systems refer to polymer solutions that contain all of the chemical components necessary to cross-link. A dual-component systems refer to polymer solutions that require chemical(s) residing in the receiving solution to cross-link. In the latter, the shape and strength of the cross-linked polymer depends primarily on the chemical properties of the solutions as opposed to the former, which relies on physical processes to initiate the cross-linking process. Hence, the constituents of template production fall into four broad categories: single-component, physically cross-linked polymer systems; single-component, chemically cross-linked polymer systems; dual-component, physically cross-linked polymers systems; and dual-component, chemically cross-linked polymer systems. In the specific embodiments, the constituents of the polymer and the receiving solutions, and the resulting templates, are pharmaceutically acceptable.

### Single-Component, Physically Cross-Linked Polymer System

In certain embodiments, a polymer solution is delivered into a single-part receiving solution, resulting in the polymer with the droplets cross-linking. The cross-linking process is physical in nature. In these embodiment, all the chemicals necessary to form a cross-linked polymer droplets reside within the polymer solution.

Polymer Solution. Several types of polymers are suitable for forming the polymer solution for a single-component, physically cross-linked polymer system. A non exclusive list includes agar, sodium alginate, calcium alginate, and sodium carboxymethyl cellulose. The above listed polymers do not need a cross-linking agent to form a gel. These polymers can undergo a transition from a liquid to a semi-solid gel upon changes in temperature or in pH.

Receiving Solution. The receiving solution can be immiscible with the polymer solution. In specific embodiments, the receiving solution is at a temperature below the gelling temperature of the polymer comprising the polymer solution. In alternative embodiments, the receiving solution is at a pH which induced cross-linking of the polymer solution.

### Single-Component, Chemically Cross-Linked Polymer Systems

Alternatively, a polymer solution can be delivered into a receiving solution, were the cross-linking mechanism is chemical in nature. In specific embodiments, photo-resist polymers are utilized. In specific embodiments, the resulting templates are cured to increase gel strength. Photo-resist polymers present very controllable non-temperature dependent cross-linked polymer systems.

Polymer Solution. Polymers for use in polymer solutions of single-component chemically cross-linked polymer systems include, but are not limited to polyethylene glycol, polydimethyl siloxane, and photo-resist polymers like SU 8, AZ-111, and polymethyl methacrylate-photoresists.

Receiving Solution. The receiving solution can be immiscible with the polymer solution. If the polymer solution comprises a non-polar organic solvent, the receiving solution can be aqueous. Once the droplet is delivered into the receiving solution a strong Ultra Violet (UV) light can be aimed at the receiving solution (in the droplet's path), resulting in the photo-resist polymer cross-linking. In specific embodiments, the same electrical pulse used to generate the droplets of the polymers is sent to a delay generator (such as an uncharged capacitor) and then to a strong UV light. In doing so, the timing of solution ejection and gel formation can be controlled by regulating the timing of the UV light.

### Dual-Component Physically Cross-Linked Polymer System

In a dual-component, physically cross-linked polymer system the polymer droplets interact with a receiving solution that contains a cross-linking agent. The polymer droplets cross-link or form upon interaction with the cross-linking agent. However, the cross-linking of the polymer droplets occur through electrostatic interactions not the formation of covalent bonds.

Polymer Solution. Several types of polymers are suitable for forming the polymer solutions for dual-component, physically cross-linked polymer systems. Examples of such polymers include, but are not limited to, sodium alginate and hydroxypropylmethylcellulose. In a specific embodiment of this invention, the composition of the polymer solution is 0.67 wt % low viscosity sodium alginate.

Receiving Solution. The receiving solution can have a hydrophobic component, a hydrophilic component, and a cross-linking agent. The cross-linking agent can be miscible with the hydrophilic component and immiscible with the hydrophobic component. The hydrophilic component can carry the cross-linking agent into the polymer droplet, resulting in the formation of a template. Examples of such mixtures include hydrocarbon-alcohol mixtures, such as the 50% heptane, 50% ethanol volume mixture containing 1.5 wt % calcium chloride exemplified below. Any of the organic solvents such as heptane, octane, nonane, or decane, or petroleum ether, can be mixed with an alcohol, such a methanol, ethanol, or propanol, provided the cross-linking agent is soluble enough in the mixture to cross-link the polymer for template formation.

Cross-linking agent in solution with miscible solvent. Cationic cross-linking agents that can be in solution with the miscible solvent include, but are not limited to, calcium chloride, magnesium chloride, calcium sulphate, and magnesium sulphate. In specific embodiments, the cross-linking agent makes a strong biocompatible gel that will degrade in the absence of the ambient cross-linking agent. For example, calcium makes a strong biocompatible gel with sodium alginate, but also diffuses away from the template to a calcium deficient environment.

### Dual-Component, Chemically Cross-Linked Systems

In a dual-component, chemically cross-linked polymer system the polymer droplets interact with a receiving solution that contains a cross-linking agent. The polymer droplets cross-links upon interaction with the cross-linking agent. However, in contrast to the physically cross-linked polymer systems, the cross-linking occurs through the formation of covalent bonds.

Polymer Solution. Several types of polymers are suitable for forming the polymer solution of a dual-component, chemically cross-linked polymer system. A nonexclusive list includes collagen (types I and II), polyvinyl alcohol, poly-L-lysine and polycationic cellulose derivatives. Examples of suitable cellulose derivatives are ethyl cellulose and reaction mixtures of partial acetate esters of cellulose with phthalic anhydride. Other examples of suitable cellulose derivatives are cellulose acetate trimellitate; methylcellulose; hydroxypropyl methyl cellulose phthalate; hydroxypropoyl methyl cellulose succinate; and polyvinyl acetate phthalate.

Receiving Solution. The two-part receiving solution can comprise a hydrophobic component, a hydrophilic component, and a cross-linking agent, e.g., as set forth above. The cross-linking agent can be miscible with the hydrophilic component and immiscible with the hydrophobic component. The hydrophilic component can carry the cross-linking agent into the polymer droplet, resulting in the formation of a template.

Cross-linking agent in solution with miscible solvent. Cross-linking agents in solution with miscible solvent include, but are not limited to, di-vinyl sulfone and 2,2-dimethoxy-2-phenylacetophenone.

### Solvent Exchange

The solvent of the templates may be exchanged before they enter the polymer bath. Volatile components can be purged from the templates by spinning the mixture in a rotary evaporator or heating the mixture. Alternatively, the templates can be separated using centrifugation or ultrafiltration. The separated templates can then be introduced to a solution and any remaining undesired solvent can be removed through gradient diffusion. Once the templates reside in the desired solvent a charge-neutral polysaccharide or polymer can be added to the suspension to match the density of the surrounding solution to that of the microspheres. This precludes collection of the microspheres at the bottom of the mixing container due to separation by weight.

Coating the Templates

In specific embodiments, the templates are coated within or infused with a polymer. The volumes of the microstructures do not appreciably change after they have been coated. The polymer coating can infuse into the template to form a stronger microstructure. For example, chitosan infusing into calcium alginate templates; the polycationic species chitosan replaces calcium as the cation source. The covalently bonded positively charged unit of chitosan forms a greater number of electrostatic interactions with any two given alginic acid chains giving rise to a microstructure with a more robust, less permeable shell and a less robust, more permeable core. A nonexclusive list of polymers for use the polymer bath includes, but is not limited to, chitosan, polycationic amino acids, such as poly-L-lysine, and polycationic cellulose derivatives. A nonexclusive list of polymers that can be utilized for template production along with a nonexclusive list of polymers that can be used to coat the templates are set-forth below in Table I.

TABLE I

| | | | Collagen | |
|---|---|---|---|---|
| | | | Derivatives | Acrylic |
| | | | (e.g., Gelatin | Polymers |
| Polymer | Calcium | Chitosan | Methylated | (e.g., Acrylic |
| Templates | Alginate | Derivatives | Collagen) | Photoresists) |
| | Chitosan | Alginate | Alginate | Cellulose |
| | | | | Derivatives |
| Shell | Other Cellulose | Collagen | Cellulose | Alginate |
| Polymers | Derivatives | Derivatives | Derivatives | |
| | (e.g., | | (e.g., | |
| | Carboxymethyl | | Carboxymethyl | |
| | Cellulose) | | Cellulose) | |
| | Polyethylene | | Polyethylene | |
| | Glycol | | Glycol | |
| | Acrylic | | | |
| | Polymers | | | |

This invention allows for varying shell thicknesses in the template. By varying the thickness of the outer polymer layer and the amount of polymer infused in the template, the release and/or absorption profile of the active agent(s) can be modulated and accurately controlled.

In general, the longer the templates reside in the polymer bath and the thicker the polymer shell formed the greater the amount of polymer that infuses into the template, the greater the radial penetration of the polymer for some given critical polymer concentration.

If a broad distribution or population of shell thicknesses is desired, the templates can have a broad distribution of residence times in the polymer bath. If a single or narrow distribution of shell thickness is desired, the templates can have identical or nearly identical residence times in the polymer bath.

### Continuous Feed Coating

The stirred polymer bath can have both inlet and outlet streams that serve as a means for the continuous introduction and removal of the templates. In a specific embodiment of the invention, a broad distribution of residence times or a population of shell thicknesses is achieved by controlling the inlet and outlet streams to have varying volumetric flowrates. In another specific embodiment of the invention, a narrow distribution of residence times or a narrow distribution of shell thicknesses is achieved by controlling the inlet and outlet streams to have constant volumetric flowrates and densities. In preferred embodiments flowrates and densities are also controlled to preserve the desired shape of the microstructures.

In another embodiment of the invention several narrow distributions of shell thickness populations are combined to form a mixture of microstructures with differing shell thicknesses.

Microcapsule Formation

In a specific embodiment of the invention, the template of a multi-layered microstructure dissolves through the introduction of an appropriate solvent. The result is the formation of a microcapsule.

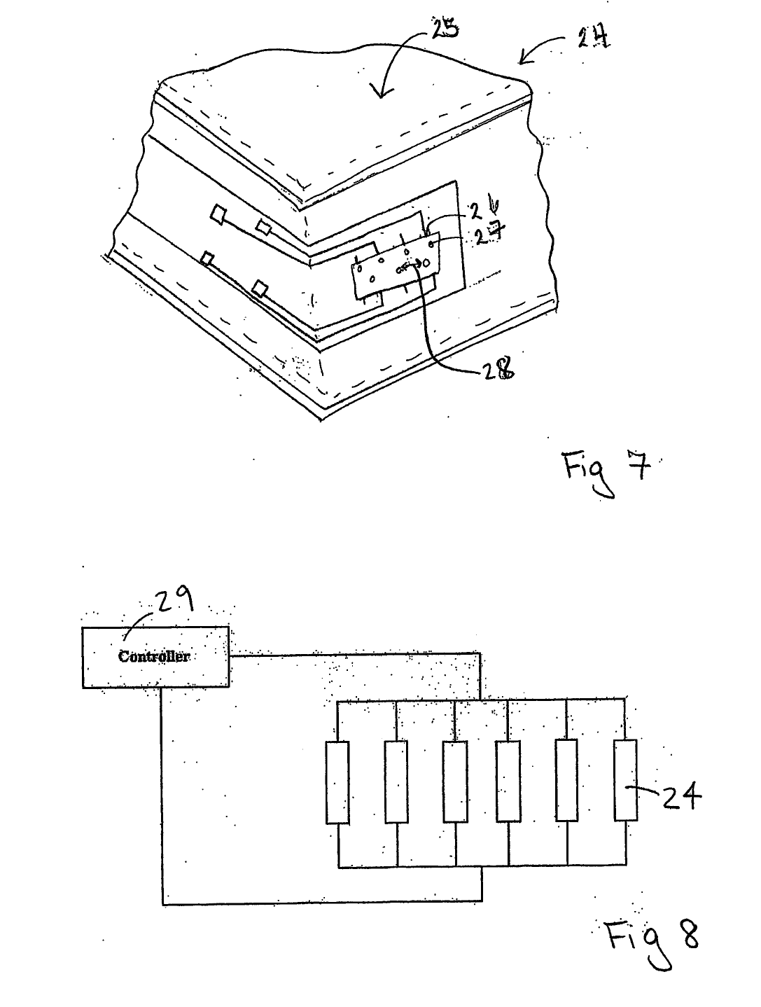

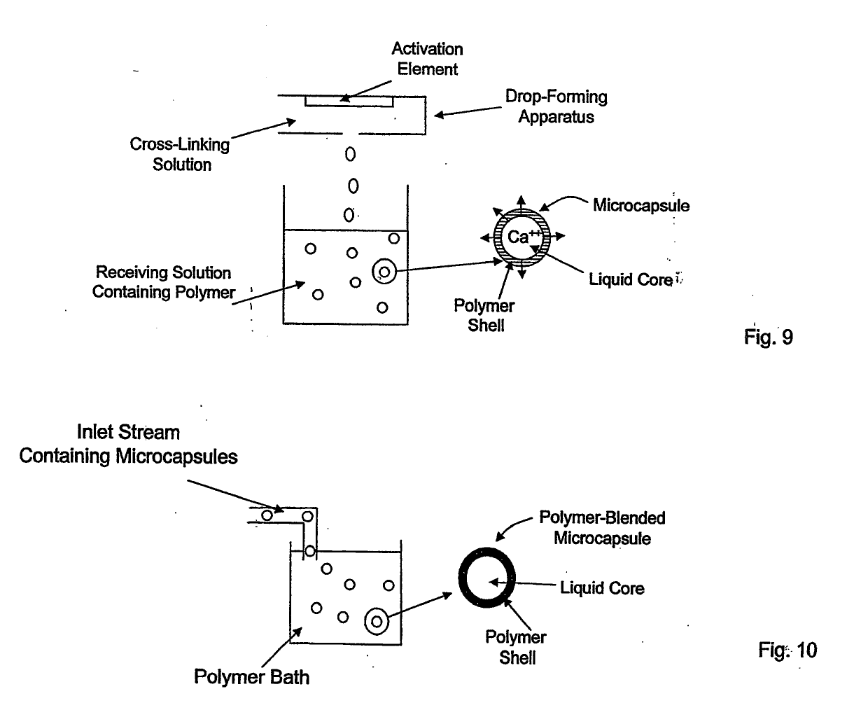

In other embodiments of this invention, microcapsules are formed without using a template. For example, the interaction of reproducible droplets containing a cross-linking agent with a receiving solution comprising a polymer results in the formation of microcapsules, FIG. 9. Once the droplets containing the cross-linking agent are received by the polymer-receiving solution, the cross-linking agent diffuses to the interface of the droplets were the polymer cross-links onto the surface of the droplet to form a membrane therearound. The concentration of cross-linking agent in the droplet can affect the thickness of the polymer shell formed, and hence the volume of the microcapsules. In general, the greater the concentration of the cross-linking agent in the droplet, the thicker the resulting shell of the microcapsule and the greater the volume of the microcapsules.

In specific embodiments, the concentration of the cross-linking agent in the droplets is varied from droplet to droplet. This variation can result in the formation of a population of microcapsules with varying volumes. The use of a secondary solvent in receiving solution that increases the interfacial tension between the droplets and the receiving solution, results in the droplets adopting a structure that minimizes contact with the receiving solution, i.e., a sphere. In specific embodiments, the secondary solvent of the receiving solution is immiscible with the cross-linking solution. The shell of the microcapsules can be blended through interaction of the microcapsules with a polymer bath, FIG. 10. Further processing can include components of a chemically cross-linked polymer being added throughout the walls of the microcapsules to control material properties or to induce functionality for site-specific delivery.

Another embodiment relates to environmentally dependent delivery of ultra-high molecular weight actives through microcapsules. A solution of an active, environmentally sensitive polymer that does not undergo ionic gelation (i.e., methacrylic copolymers, block copolymers based on ethylene oxide and propylene oxide, etc.), and a physically cross-linkable hydrogel polymer (i.e., alginic acid, collagen, etc.) is subjected to a solution containing a physical cross-linking agent (i.e., in the case of alginic acid, a solution rich in divalent cationic species). The resultant physically cross-linked gel is chosen to have a molecular mass cut-off lower than that of the environmentally sensitive polymer such that the environmentally sensitive polymer is physically trapped in the hydrogel microsphere, yet remains in solution. After the hydrogel template containing the environmentally sensitive polymer has formed, the ambient conditions are altered (e.g., in the case of methacrylic copolymers, the solution is made more acidic) to cause the environmentally sensitive polymer to leave solution, forming an environmentally sensitive layer that grows inward toward the core of the microsphere as more of the pH sensitive polymer is exposed to the ambient conditions. Once the desired layer thickness is achieved, ambient conditions are changed once again to halt growth of the environmentally dependent layer and simultaneously dissolve the physically cross-linked polymer scaffold, thereby forming environmentally sensitive fluid-core microcapsules. Much as the effective permeability of the polymer shell can be controlled in vitro, environmental cues can be used to control release in vivo. Additionally, in the case of sono-sensitive polymers, microcapsules pharmacokinetics can be cued by external factors such as ultrasonic vibration.

### Drop-Forming Apparatus

The disclosed drop-forming apparatus for loaded microstructures can be utilized for the manufacturing of microcapsules without using a template. The same factors that affect reproducible single-droplet generation of polymer droplets can affect reproducible single-droplet generation of droplets containing the cross-linking agent. Additionally, the concentration of the cross-linking agent can be varied from droplet to droplet, resulting in the formation of a population of microcapsules with varying volume.

### Cross-Linking & Polymer-Receiving Solutions

Dual-component polymer systems can be used to form microcapsules without using a template. The cross-linking mechanism of these systems can be physical or chemical in nature. The cross-linking agent is contained in the cross-liking solution, which is loaded into the drop-forming apparatus. The concentration and the charge density of the cross-linking agent can affect the volume and strength of the microcapsules.

Cross-linking Solution. Several types of cross-linking agents are suitable for forming the cross-linking solution. A nonexclusive list includes cellulose derivates, calcium chloride, and magnesium chloride. The cross-linking solution can contain a hydrophobic component, a hydrophilic component and a cross-linking agent. The cross-linking agent can be miscible in the hydrophobic component.

Polymer-Receiving Solution. The polymer-receiving solution can comprise of a hydrophobic component and a polymer. A nonexclusive list of polymers that can comprise the polymer-receiving solution include, but are not limited to, alginate and cellulose derivates.

### Blended Microcapsules

In specific embodiments, the microcapsules are blended by the interaction of the microcapsules with a polymer bath as shown in FIG. 9. The introduction of microcapsules into a polymer bath can result in a polymer diffusing into the shell of the microstructure and replacing the cross-linking agent. The blended microcapsule can have a greater mechanical strength than its non-blended counterpart. Unlike the microstructures, the residence time of the microcapsules in the polymer bath does not affect the volume of the structures.

Active Agent Loading

In this invention, the back-loading of the active agent(s) into the microstructures is usually diffusion controlled. Generally the microstructures are separated from the polymer bath and introduced into a concentrated active agent solution. Active agent uptake will vary, depending upon the ratio of the components employed and on the particular active involved. The loading capacity of a microstructure can be augmented by the introduction of an appropriate solvent to dissolve the core or template. The proportional ratio of active agent to carrier naturally depends on the chemical nature, solubility, and stability of the compositions, as well as the dosage contemplated. In certain specific embodiments (i.e., where the active agent is insulin), the drug content of the microstructures, by weight, may be from about 0.2 to about 1%.

A number of active agents can be released in a controlled method in this invention. These include, but are not limited to, small molecules, nutrients, flavorants; and macromolecular compounds such as polypeptides, proteins, hormones, and nucleic acid materials comprising DNAs and antisense molecules. In specific embodiments, the active agents have a molecular weight in the range of about 5 to about 25 kDa and are soluble in aqueous media.

### Proteins

A nonexclusive list of proteins and peptides that can be used as the active component in this invention includes: erythropoietin (EPO), granulocyte colony stimulating factor, ganulocyte monocyte colony stimulating factor, interferon alpha, interferon beta, oxytocin, captopril, bradykinin, atriopeptin, cholecystokinin, heparin endorphins, nerve growth factor, melanocyte inhibitor-I, gastrin antagonist, somatotatin, encephalins growth hormone, insulin, insulin-like growth factors, and the like. Both recombinant and natural protein and peptide product can be used.

### Nutrients

Suitable nutrients include, but are not limited to, vitamins, amino acids and derivatives thereof and minerals. Examples of such nutrients include vitamin B complex, thiamine, nicotinic acid, biotin, pantothenic acid, choline riboflavin, vitamin B6, vitamin B12, pyridoxine, insositol, carnitine, ascorbic acid, ascorbyl palmitate, vitamin A and its derivatives (vitamin A alcohol, vitamin A esters, vitamin A aldhyde), vitamin K, vitamin E, vitamin D, cysteine and N-acetyl cysteine, herbal extracts, and derivatives thereof.

### Nucleic Acids

Nucleic acids may be released as the active agent in the controlled method of this invention. The term nuclei acid includes deoxyribonucleic acid (DNA) and ribonucleic acid (RNA). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, is uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups.

The present invention is particularly useful for administering oligonucleotides, e.g., anti-sense, ribozyme, and RNAs oligonucleotides.

### Hormones

A nonexclusive list of hormones that can be administered according to the invention include progestins (progestogens), estrogens, thyrotropin-releasing hormone (TRH), vasopressin, gonadotropin-releasing hormone (GnRH or LHRH), melanotropin-stimulating hormone (MSH), calcitonin, growth hormone releasing factor (GRF), parathyroid hormone, and the like.

### Small Molecules

Small molecules that can be utilized as the active agent in the present invention include, but are not limited to, 1) antipyretic analgesic anti-inflammatory agents such as indomethacin, aspirin, diclofenac sodium, ketoprofen, ibuprofen, mefenamic acid, dexamethasone, dexamethasone sodium sulfate, hydrocortisone, prednisolone, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; 2) antiulcer agents such as ranitidine, sulpiride, cetraxate hydrochloride, gefarnate, irsogladine maleate, cimetidine, lanitidine hydrochloride, famotidine, nizatidine or roxatidine acetate hydrochloride; 3) coronary vasodilators such as Nifedipine, isosorbide dinitrate, diltiazem hydrochloride, trapidil, dipyridamole, dilazep dihydrochloride, methyl 2,6-dimethyl-4-(2-nitrophenyl)-5-(2-oxo-1,3,2-dioxaphosphorinan-2-yl)-1,4-dihydropyridine-3-carboxylate, verapamil, nicardipine, nicardipine hydrochloride or verapamil hydrochloride; 4) peripheral vasodialtors such as ifenprodil tartrate, cinepazide maleate, cyclandelate, cinnarizine or pentoxyfyline; 5) oral antibacterial and antifungal agents such as penicillin, ampicillin, amoxicillin, cefalexin, erythromycin ethylsuccinate, bacampicillin hydrochloride, minocycline hydrochloride, chloramphenicol, tetracycline, erythromycin, fluconazole, itraconazole, ketoconazole, miconazole or terbinafine; 6) synthetic antibacterial agents such as nlidixic acid, piromidic acid, pipemidic acid trihydrate, enoxacin, cinoxacin, ofloxacin, norfloxacin, ciprofloxacin hydrochloride, or sulfamethoxazole triinethoprin; 7) antispasmodics such as popantheline bromide, atropine sulfate, oxapium bromide, timepidium bromide, butylscopolamine bromide, rospium chloride, butropium bromide, N-methylscopolamine methylsulfate, or methyloctatropine bromidebutropium bromide; 8) antitussive, anti-asthmatic agents such as theophylline, aminophylline, methylephedrine hydrochloride, procaterol hydrochloride, trimetoquinol hydrochloride, codeine phosphate, sodium cromoglicate, tranilast, dextromethorphane hydrobromide, dimemorfan phosphate, clobutinol hydrochloride, fominoben hydrochloride, benproperine phosphate, tipepidine hibenzate, eprazinone hydrochloride, clofedanol hydrochloride, ephedrine hydrochloride, noscapine, calbetapentane citrate, oxeladin tannate, or isoaminile citrate; 9) broncyodilators such as diprophylline, salbutamol sulfate, clorprenaline hydrochloride, formoterol fumarate, orciprenalin sulfate, pirbuterol hydrochloride, hexoprenaline sulfate, bitolterol mesylate, clenbuterol hydrochloride, terbutaline sulfate, mabuterol hydrochloride, fenoterol hydrobromide, or methoxyphenamine hydrochloride; 10) diuretics such as furosemide, acetazolamide, trichlormethiazide, methyclothiazide, hydrochlorothiazide, hydroflumethiazide, ethiazide, cyclopenthiazide, spironolactone, triamterene, fluorothiazide, piretamide, metruside, ethacrynic acid, azosemide, or clofenamide; 11) muscle relaxants such as chlorphenesin carbamate, tolperisone hydrochloride, eperisone hydrochloride, tizanidine hydrochloride, mephenesin, chlorozoxazone, phenprobamate, methocarbamol, chlormezanone, pridinol mesylate, afloqualone, baclofen, or dantrolene sodium; 12) brain metabolism altering drugs such as meclofenoxate hydrochloride; 13) minor tranquilizers such as oxazolam, diazepam, clotiazepam, medazepam, temazepam, fludiazepam, meprobamate, nitrazepam, or chlordiazepoxide; 14) major tranquilizers such as Sulpirid, clocapramine hydrochloride, zotepine, chlorpromazinon, or haloperidol; 15) β-blockers such as pindolol, propranolol hydrochloride, carteolol hydrochloride, metoprolol tartrate, labetalol hydrochloride, acebutolol hydrochloride, butetolol hydrochloride, alprenolol hydrochloride, arotinolol hydrochloride, oxprenolol hydrochloride, nadolol, bucumolol hydrochloride, indenolol hydrochloride, timolol maleate, befunolol hydrochloride, or bupranolol hydrochloride; 16) antiarrhythmic agents such as procainamide hydrochloride, disopyramide, ajimaline, quinidine sulfate, aprindine hydrochloride, propafenone hydrochloride, or mexiletine hydrochloride; 17) gout suppressants allopurinol, probenecid, colchicine, sulfinpyrazone, benzbromarone, or bucolome; 18) anticoagulants such as ticlopidine hydrochloride, dicumarol, or warfarin potassium; 19) antiepileptic agents such as phenyloin, sodium valproate, metharbital, or carbamazepine; 20) antihistaminics such as chlorpheniramine maleate, cremastin fumarate, mequitazine, alimemazine tartrate, or cycloheptazine hydrochloride; 21) antiemetics such as Difenidol hydrochloride, metoclopramide, domperidone, betahistine mesylate, or trimebutine maleate; 22) hypotensives such as dimethylaminoethyl reserpilinate dihydrochloride, rescinnamine, methyldopa, prazosin hydrochloride, bunazosin hydrochloride, clonidine hydrochloride, budralazine, or urapidin; 23) sympathomimetic agents such as dihydroergotamine mesylate, isoproterenol hydrochloride, or etilefrine hydrochloride; 24) expectorants such as bromhexine hydrochloride, carbocysteine, ethyl cysteine hydrochloride, or methyl cysteine hydrochloride; 25) oral antidiabetic agents such as glibenclamide, tolbutamide, or glymidine sodium; 26) circulatory agents such as ubidecarenone or ATP-2Na; 27) iron preparations such as ferrous sulfate or dried ferrous sulfate; 28) vitamins such as vitamin B1, vitamin B2, vitamin B6, vitamin B12, vitamin C, vitamin A, vitamin D, vitamin E, vitamin K or folic acid; 29) pollakiuria remedies such as flavoxate hydrochloride, oxybutynin hydrochloride, terodiline hydrochloride, or 4-diethylamino-1,1-dimethyl-2-butynyl (I)-α-cyclohexyl-α-phenylglycolate hydrochloride monohydrate; 30) angiotensin-converting enzyme inhibitors such as enalapril maleate, alacepril, or delapril hydrochloride; 31) anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine; 32) high potency analgesics such as codeine, dihydrocodeine, hydrocodone, morphine, dilandid, demoral, fentanyl, pentazocine, oxycodone, pentazocine or propoxyphene; 33) antihistamines such as Brompheniramine maleate and 34) nasal decongestants such as phenylpropanolamine HCl. Active ingredients in the foregoing list may also have beneficial pharmaceutical effects in addition to the one mentioned.

Formulations

A composition of this invention may be provided in a variety of physical forms. In specific embodiments, the loaded microstructures are concentrated before formulation. The different formulation techniques in this invention include, but are not limited to, lyophilization, suspensions, matrix incorporation, enteric or other coatings.

A formulation of the invention can contain other components in addition to the microstructures to further stabilize the drug. Examples of such components include, but are not limited to, carbohydrates and sugars, such as trehalose, glucose, dextrose; medium to long chain polyols, such as glycerol, polyethylene glycol, and the like; other proteins; amino acids; nucleic acids; chelators; proteolysis inhibitors; preservatives; and other components. In specific embodiments, any such constituent of a composition of the invention is pharmaceutically acceptable.

### Lyophilization

In the freeze-drying technique, the drug loaded microstructures are dissolved in an appropriate solvent. This mixture is then frozen followed by sublimating the solvent under vacuum and under supply of heat of sublimation while continuously removing the vapor formed. The resulting freeze-dried amorphous solid may be subjected to a secondary drying process at elevated temperature.

### Suspensions

In the suspension technique, drug-loaded microstructures are suspended in a suspending agent. The suspending agent can be liquid or a gel. Suspending agents include, but are not be limited to, ethoxylated isosterayl alcohols, polyoxyethylene sorbitol and sorbitan esters, microcrystalline cellulose, aluminum methahydroxide, bentonite, agar-agar and tragacanth, or mixtures of these substances.

### Matrix Incorporation

In the matrix incorporation technique, the loaded microstructures are distributed evenly through a matrix polymer, whereby active agent released from the microstructures is released from the matrix as a result of diffusion and/or polymer erosion.

### Enteric or Other Coating

In the enteric coating technique, a finite number of drug-loaded microstructures are encapsulated in a single larger sphere comprising of substance which does not dissolve in the acid environment of the stomach but does dissolve in the alkaline environment of the small intestines, hence allowing for release of the drug there. Constituents that would comprise the enteric coatings include, but are not limited to, hydroxypropylmethylcellulose phthalate, methacrylic acid-methacrylic acid ester copolymer, polyvinyl acetate-phthalate, methacrylic copolymers, and cellulose acetate phthalate.

In addition, other coatings may be used to target the release the active agent at various regions in the body. The coatings may be a single layer or multiple layers. The “coating weight,” or relative amount of coating material per dosage form, generally dictates the time interval between ingestion and initial drug release. The encapsulated structures can contain the loaded microstructures in addition to the customary excipients, such as fillers and extenders, binders, humectants, disintegrating agents, solution retarders, absorption accelerators, wetting agents, adsorbents, and lubricants.

### Microcapsule Functionalization Techniques

Functionalization of the capsule wall for the purpose of site specific immobilization, and subsequent delivery of an active contained within, can be achieved by methods ranging from chemical conjugation of linking groups containing biochemical receptors to the capsule surface to the introduction of functional polymers, entangled throughout the polymer capsule's constitution. Several of these physical and chemical immobilization schemes are described below:

### Chemical Conjugation

Functional groups naturally present or evolved, on the surface of one or more of the capsule's constituent polymers are to be joined with multifunctional linking groups specific to some material to which the capsule is to link. These groups are chemically fundamental in nature (i.e., amine, hydroxyl, carboxylic acid groups) and allow for an anchoring of larger, often more complex linking groups, ranging from synthetic linkers like multifunctional polyethylene glycol (PEG) to biochemical linkers such as the biotin/streptavidin/biotin-substrate linking complex. In each case, oxidation or reduction of the polymer capsule surface at the aforementioned functional loci is involved. The surfaces can be modified to create functionality by such means as exposure to UV light or cold plasmas to create free radical groups which can react to form amine, hydroxyl, or carboxylic acid groups or exposure to liquid chemical media such as ethylene diamine to promote aminolysis and the like, of the polymer substrate.

### Physical Functionalization

Physical entanglement. Functional polymers, that promote preferential binding to a substrate or non-specific binding by way of Coulombic or weak chemical interaction, can be introduced into the capsule wall either by way of diffusion or incorporation into the medium expelled from the ERS CG. In either case, physical entanglement of the polymer and attractive interaction between the physically entangled polymers is used to immobilize the functional polymer, at least some of which will be at the polymer capsule surface, for the purpose of promoting binding, either chemically or by physical interaction to a substrate to immobilize the capsule.

Macromolecular anchoring. Ultra-high-molecular-weight molecules or metallic/ceramic nanoparticles can be used to anchor a functional polymer to the body of the capsule, at least some of which is to be exposed at the capsule surface. Chemical conjugation to a non-functional “anchor” above the molecular mass cutoff of the polymer blend from which the capsule is made allows for immobilization of the functional polymer at the capsule surface, even when no modification of the capsule's constituent polymers is feasible for the purpose of functionalization for the sake of site specific immobilization of the body of the capsule.

In one embodiment, conjugated IgG2 antibodies can be front-loaded into microspheres. By conjugating the IgG2 antibodies before gelation, the cumulative molecular weight can be made sufficiently large so as to immobilize the conjugated IgG2 antibodies within the gel. The terminal IgG2 antibody can be conjugated to any IgG1 protein-specific antibody to achieve protein specific binding between the microsphere and a protein of choice.

Therapeutic Delivery of Active Agent

The invention enables facile modulation of the pharmacokinetic/pharmacodynamic profile of an active agent because of the high degree of control provided over the timing and rate of drug release. The critical factors that affect the release rate of the active agent are the thickness of the polymer coating or shell or the amount of the polymer that has infused into the template, and the permeability of the template and polymer shell.

### Sigmoidal Release

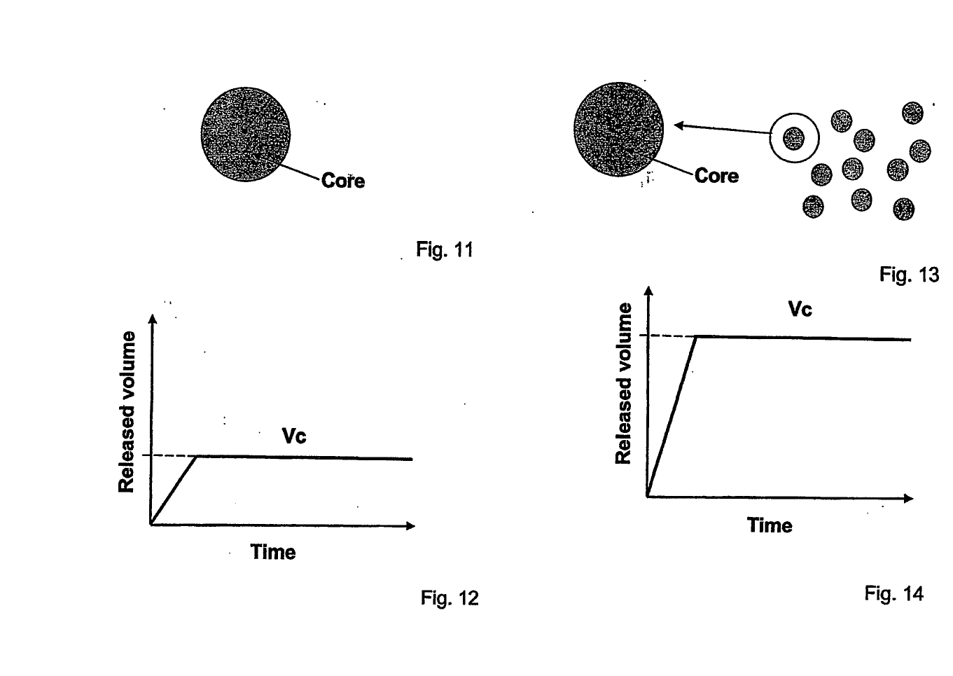

In general, the particular release profile for any given spherical microstructure is sigmoidal in nature. By “sigmoidal in nature” is meant any function that contains two plateaus, an initial and a final, joined by a region of release that can be approximated as linear. In specific embodiments, a microsphere comprises a loaded template with no additional coating layer. The template can have an in vitro release profile containing one plateau corresponding to the global time to release. A diagram of a template is shown in FIG. 11. A corresponding release profile of the template is shown in FIG. 12.

In alternative embodiments, a population of templates shown in FIG. 13 can have an in vitro release profile shown in FIG. 14. The release profile shown in FIG. 14 contains one plateau, however, the slope of the linear region immediately preceding the plateau has increased. In general, increasing the number of templates with a given active agent uptake, increases the slope of the linear region between plateaus or immediately preceding plateaus in the in vitro release profile.

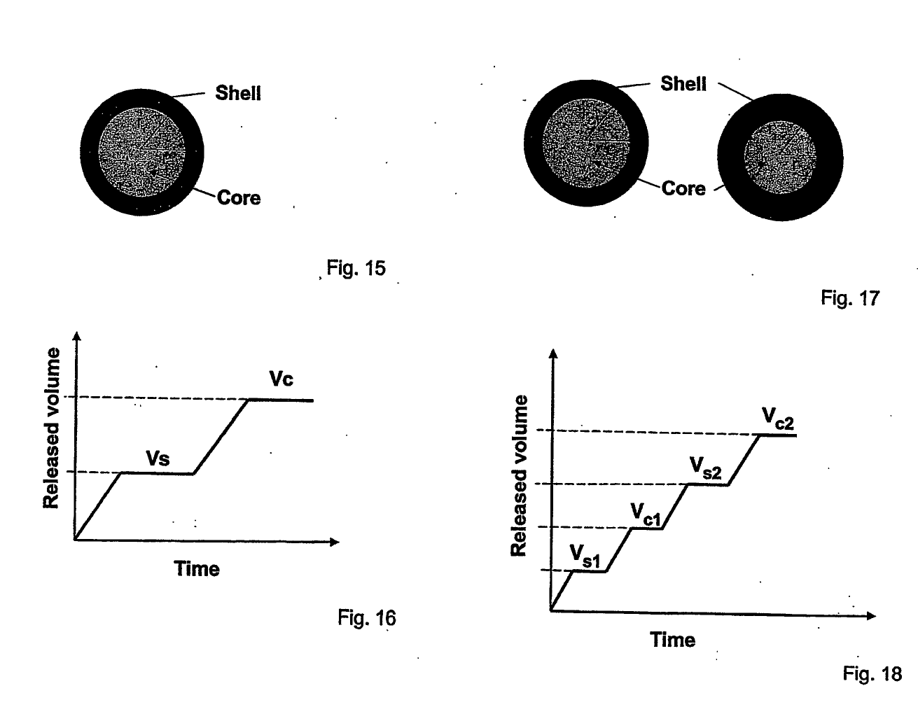

The wall thickness of the outer polymer layer is responsible for the difference in time to maximum release from microstructure to microstructure. In specific embodiments, the active agent is present in both the outer wall and in the core of the microstructure (FIG. 15), the in vitro release profile contains two plateaus corresponding to the two release maxima (FIG. 16). The first release maximum corresponds to the time to maximum release of the outer wall contents. The second release maximum, which marks the global time to release, corresponds to the time to release of the core contents.

### Complex Release

In general, varying wall thicknesses allows for increasing (in the case of incrementing wall thickness) or decreasing (in the case of decrementing wall thickness) the time to maximum release, while decreasing or increasing the slope of the linear region between the sigmoidal plateaus, respectively. Complex release can be obtained by a population of microstructures with varying wall thicknesses.

The release profile of a mixture comprising two loaded microstructures with varying shell thicknesses is shown in FIG. 18. The microstructures can have the same volume, but differ in the thickness of their shells, FIG. 17. The release profile of the mixture of the microstructures can contain four plateaus, FIG. 18. The first two plateaus can correspond to the time to maximum release of the outer wall and the inner core of the microstructure comprising a thinner shell, respectively. The final two plateaus can correspond to the time to maximum release of the outer wall and the inner core of the microstructure contains the thicker shell, respectively.

By extrapolating the results for the mixture of two loaded microstructures, it can be seen that by varying the effective difference in wall thickness from microstructure to microstructure in a population comprising of hundred of millions of microstructures, any desired increasing in vitro release profile can be obtained.

### Translate into Pharmacokinetics: Release Profile that Mimics Circulatory or Daily Levels

In another embodiment, release profiles that mimic circulatory or daily levels in the human body are obtained. In these specific embodiments, the “consumption function” and desired in vivo release can be known. By “consumption function” is meant the quantification of the mechanism by which the body removes an active agent(s) from the body. The consumption function has been determined and is available when engineering release systems for a number of active agents.

As discussed earlier, this invention allows for the easy formulation of any desired increasing in vitro release profile by engineering microstructures with specific continuously varying wall thicknesses. The combination of the in vitro release profile and the consumption function allows for prediction of the how the active agent is addressed by the body. In order to engineer microstructures so as their in vivo release profiles closely match the natural cycles of certain human-derived, biological macromolecules in individuals, the consumption function can be subtracted from the desired in vivo release profile. The result of this subtraction is the desired in vitro release profile. Any desired increasing in vitro release profile can be obtained as described above. Hence, loaded microstructure can be engineered so as their in vivo release profiles closely match the natural cycles of certain human-derived, biological macromolecules in individuals.

“Burst” Release

In yet another embodiment of the invention, the release of the active agent from the microstructures is violent and sudden. For example, microstructures having a hydrophobic core immediately burst when placed in a water-rich environment. The templates can be swollen so that the contained fluid exerts tensile stress on the template. The template can expel its contents and then relax to its equilibrium configuration.

Transdermal

Alternatively, a transdermal formulation form can be utilized. Transdermal formulations may be a diffusion transdermal system (transdermal patch) using either a fluid reservoir or a drug-in-adhesive matrix system. Other transdermal formulations include, but are not limited to, topical gels, lotions, ointments, transmucosal systems and devices, and iontophoretic (electrical diffusion) delivery systems.

Personalized Formulations for Individual Pharmacokinetic and Pharmacodynamic Profiles

In another embodiment of this invention, the polymer shell thicknesses of the microstructures are modulated to obtain personalized formulations for individual pharmacokinetic and pharmacodynamic profiles. This is a useful improvement because it permits the physician to recommend with a high degree of certainty, a dosage that will have the predicted pharmacokinetic profile for a patient.

For example, it is well known that there is substantial interindividual variability in the pharmacokinetics of many antileukemic agents in children (Ching-Hon, Pui; Childhood Leukemias; Cambridge: University Press (1999)) and that interindividual differences in the pharmacokinetics of antileukemic agents can affect the efficacy and toxicity of antileukemic therapy (Ching-Hon, Pui; Childhood Leukemias; Cambridge: University Press (1999)). Consequently, tailoring the dosage of antileukemic agents for children will increase the efficacy of the active agent.

### Microcapsule Dimensional Response

Microcapsules are significantly affected by their ambient environment. Small changes in the ambient salt concentration and water content have a large impact on the overall microcapsule volume. These volumetric changes can be greater than an order of magnitude. When the ambient receiving solution is exchanged for double-distilled water, the resultant population of microcapsules experiences a change in volume via a solvent-exchange mechanism.

Cell Encapsulation

In specific embodiments, a single cell is contained in the liquid core of a microcapsule. Single-cell encapsulation can be achieved utilizing either of the above disclosed methods for microcapsule formation.

For example, reproducible volume droplets of a polymer-cell suspension are introduced into a receiving solution that cross-links the droplets, such that each droplet contains, on average one cell. An outer polymer shell can be formed through interaction of the templates with a polymer bath, just as with the microstructures. The templates can be dissolved by the introduction of an appropriate solvent, resulting in encapsulated cells. The microcapsules comprising live cells are separated from those which contain dead cells or no cells. The encapsulated cells can then be coated to enable site-specific targeting, as discussed above for the microstructures.

Alternatively, single-cell encapsulation can be achieved without utilizing a template. For example, a reproducible volume droplets of a suspension comprising cells and a cross-linking agent are introduced into a polymer-receiving suspension, such that each droplet contains, on average one cell. Once the droplets are received into the polymer-receiving solution the cross-linking agents diffuses to the interface of the droplet and the polymer cross-links on surface of the droplet. The interaction of microcapsules whose liquid cores contain cells with a polymer bath results in a polymer-blended cell containing microcapsules. The microcapsules comprising live cells are separated from those comprising dead cells or no cells. The encapsulated cells can then be coated to enable site-specific targeting, as discussed above for the microstructures.

Once the encapsulating matrix is introduced into the blood stream, the matrix can be dissolved or removed (by some internal immune process such as macrophage phagocytosis) before it encounters the liver or the kidneys. In an alternative embodiment, the encapsulating matrix can be confined to a region of the body where the matrix is prevented from freely circulating.

### Cells

Cells of various shape and volume can be encapsulated in this invention. To prepare cells for ejection from the nozzles of the drop-forming apparatus, cells can be cultured. In a specific embodiment, cells are cultured, filtered, pelleted, and then suspended in the polymer solution. A nonexclusive list of cells that can be encapsulated by this invention include insulin bovine and porcine b-pancreatic islet cells.

### Polymer & Receiving Solutions

Polymer and receiving solution combinations for the formation of templates for the microstructures that can be utilized to encapsulate single cells must be compatible with the cells. For example, in order to maintain the greatest cell viability the pH, osmolarity and temperature of the polymer and receiving solutions can be matched to those acceptable to the cell. For example, harsh cross-linking agents such as ultra-violent light and nocuous chemical such as divinyl sulfone should be avoided. In general, physically cross-linked polymer systems tend to be less detrimental to cell viability than chemically-linked polymer systems and hence are preferred. Additionally, the cross-linked polymer can be reversible. For example, a gelled agar cell droplet or template will liquefy upon the introduction of agarase. Polymer systems of the invention can contain other additives such as lyophilized sheep's blood, minimal medial, or bacterial growth inhibitors to further stabilize the cells.

Cell Suspensions. In specific embodiments, cells are suspended in the polymer or cross-linking solution resulting in a cell suspension. In specific embodiments, the physiological pH, temperature, and solution osmolarity of the cell suspensions are monitored and maintained so that the cell's surrounding environment is favourable for cellular metabolism. Physiological pH can be achieved through buffering, temperature can be regulated by the addition of heaters/coolers to the body of the drop-forming apparatus, and osmolarity can be maintained by the adjustment of concentrations utilizing an inert substitute for bodily electrolytes. In specific embodiments, the inert substitute for bodily electrolytes, such as sucrose, trehalose, fructose, glucose, and mannose, do not significantly increase the viscosity of the mixture.

Primary cells, such as pancreatic B-Islet cells harvested from cadavers, or cell lines can be encapsulated. In addition, cells or cell lines selected or genetically engineered to produce a desired product, whether a protein like insulin or, the case of yeast or bacterial cells, as antibiotic compound, can be encapsulated and introduced into a host organism.

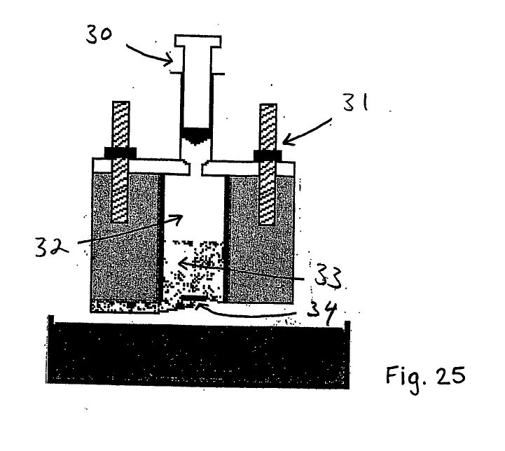

Inkjet cartridge setup. In a specific embodiment of this invention for ejecting cells from a HP 51625A inkjet cartridge, a modified inkjet cartridge is used as shown in FIG. 25. A syringe 30 is attached to a chamber that is threaded and bolted 31 to a heating and cooling chamber 32 containing the cell suspension 33. The suspension is agitated by a rice-grain sized stir bar 34 at the bottom of the cell suspension chamber.

### Targeting Microstructures

As mentioned above, the microstructures of the invention, particularly those containing a cell or cells, can be targeted in vivo using specific targeting molecules cross-linked to the shell or coat of the microstructure. Targeting molecules include, but are not limited to, antibodies (including full length immunoglobulins and Fv fragments thereof), receptor ligands, soluble receptors, carbohydrates, lectins, peptides, and other molecules that specifically bind to cells or extracellular structures. Tumor antigens represent a specific class of targets for microstructures loaded with chemotherapeutic agents.

For example, polyvinyl chloride resin microparticles, as components of a more complex microstructures, can be functionalized to allow for the building of peptide chains, one amino acid at a time. This functionalization process, which is similar to microscale Merrifield Synthesis, can be particularly useful for thrombus-specific active agents such as heparin.

The targeting molecules can be linked to the microstructures using conventional reagents. For example, one could employ conventional crosslinking agents such as carbodiimides. Examples of carbodiimides are 1-cyclohexyl-3-(2-morpholinyl-(4-ethyl)carbodiimide (CMC), 1-ethyl-3-(3-dimethyaminopropyl)carbodiimide (EDC) and 1-ethyl-3-(4-azonia-44-dimethylpentyl)carbodiimide.

Examples of other suitable crosslinking agents are cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homobifunctional crosslinking agents include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamide)ethyl]disulfide; the bifunctional aldehydes formaldehyde, malondialdehyde, succinaldehyde, glutaraldehyde, and adiphaldehyde; a bifunctional epoxied such as 1,4-butaneodiol diglycidyl ether, the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri)2-chloroethyl)amine, respectively.

Examples of other common heterobifunctional crosslinking agents that may be used to effect the conjugation of proteins to peptides include, but are not limited to, SMCC succinimidyl-4-(N-maleimidomethyl)cyclohexane-1-carboxylate), MBS (m-maleimidobenzoyl-N-hydroxysuccinimide ester), SIAB (N-succinimidyl(4-iodacteyl)aminobenzoate), SMPB (succinimidyl-4-(p-maleimidophenyl)butyrate), GMBS (N-(□-maleimidobutyryloxy)succinimide ester), MPHB (4-(4-N-maleimidophenyl)butyric acid hydrazide), M2C2H (4-(N-maleimidomethyl)cyclohexane-1-carboxyl-hydrazide), SMPT (succinimidyloxycarbonyl-á-methyl-á-(2-pyridyldithio)toluene), and SPDP (N-succinimidyl 3-(2-pyridyldithio)propionate).

Crosslinking may be accomplished by coupling a carbonyl group to an amine group or to a hydrazide group by reductive amination.

### EXAMPLES

The present invention will be better understood by reference to the following examples, which are provided by way of illustration and are not limiting.

### Example 1

### Investigation of Inkjet, Polymer Solution, and Receiving Solution Properties

Each droplet of ink expelled from a nozzle on an inkjet printhead results from the printer sending an electrical pulse (that is, applying a transient voltage across) some heating element and/or piezoelectric component of an inkjet cartridge. By emulating this electrical signal, modified inkjet cartridges have been made to eject droplets of nearly any solution with exceptional reproducibility in droplet volume. The solutions of interest are those that yield polymer hydrogels.

Polymer hydrogels exhibit a range of interesting properties including extraordinary biocompatibility and controllable permeability (or conversely “stopping power”). Hydrogels are especially interesting candidates for the controlled release of pharmaceutical agents.

By using inkjet cartridges to form hydrogel microstructures, we have developed an extremely reproducible protocol for mass-producing micro-sized structures.

a) Reverse Engineering and Reengineering of an Inkjet Cartridge

Inkjet cartridges are, fundamentally, micro-electromechanical droplet generating devices containing arrays of actuators that draw power from an electrical pulse and either generate heat or induce a mechanical (dimensional) response that expels a droplet through a pore. Using a Micronta Digital Auto-Range Multimeter the resistance across pairs of electrical contacts of an HP51625A Color Inkjet Cartridge were measured in order to determine how the electrical contacts are connected to the arrays of actuators that cause droplet ejection. The smallest non-zero resistance measured across any pair of pads was 33 Ohms. Pairs of pads registering a 33 Ohm resistance correspond to leads connected to a single actuator. Wire leads were then soldered to a pair of electrical contacts attached to a single actuator in order to systematically investigate how pulse amplitude, duration, and frequency affect the reproducibility of single droplet generation.

By back calculating based upon printing speed, droplet volume, and a few other measurable parameters, we determined that inkjet printers utilizing the HP51625A cartridge produce thousands of droplets each second. In other words they operate in the kilohertz range. Therefore, the longest single pulse is on the order of milliseconds. To determine the appropriate pulse width and amplitude (the factors that affect heating), systematic experimentation was conducted with pulse widths ranging from 0.1 microseconds to 1 millisecond by order of magnitude, and between 5 and 25 Volts. Pulse geometry was chosen to be a square wave to minimize power consumed when generating a droplet (by avoiding ramp-heating).

An HP8112A Pulse Generator and an HP Harrison 6204B DC Power Supply were wired to the leads of the inkjet cartridge as shown in FIG. 5. Frequency and pulse width were determined by the settings of the Pulse Generator and the Power Supply determined the signal amplitude (voltage). Frequency was fixed at 100 Hz to conduct initial trials to determine the relationship among pulse width, amplitude, and resultant droplet volume. When pulse width ranged from 0.1 to 1 milliseconds and amplitude ranged from 17 to 25 Volts, the foil of the cartridge became scalding to the touch and droplets were ejected in fits and spurts with intermittent periods of ink misting. When pulse width ranged from 0.1 to 1 microseconds and amplitude ranged from 5 to 10 Volts, no droplets were ejected. Qualitatively, results can be explained by consideration of the dimensional response of the actuating element and its resultant perturbation of the interfacial tension of ink/air interface at each pore. In the case in which the actuator receives too weak (too short in duration, or too low in amplitude) a pulse, the surface energy of the ink is too great for the mechanical energy driving fluid out of the pore to overcome, so no fluid is expelled. When the pulse is too strong, enough fluid is ejected such that Rayleigh instability will cause formation of multiple droplets from a single pulse, while the resistive element does not have time to recover before it is pulsed again, which is why the foil heats up tremendously.

From these surveys, it was determined that a 14-16 V square-wave pulse for 8-10 microseconds produces a single droplet, drawing approximately 1/16 Amp of current. Shorter pulses, lower currents, and smaller voltages did not allow the system to generate the heat necessary to eject a droplet. Longer pulses, higher currents, and larger voltages caused the cartridges to expel too much fluid from each pore, leading a single pulse to produce two droplets of different sizes, a primary droplet, and a secondary smaller, tailing droplet, as the result of improperly checked fluid displacement and Rayleigh instability.

To derive the relationship between the electrical contacts and the position of the actuators in relation to the pores, an inkjet cartridge was positioned over a glass slide sitting on the stage of a CKX41 Olympus Inverted Microscope. While the electronics pulsed, the leads were transferred among all of the pairs of pads and the droplet ejection was viewed at 40× magnification, noting which electrical pads corresponded to which set of nozzles.

From this study, it was determined that two pairs of pads would send pulses to the most optimal arrangement of our actuators situated over their respective pores. This combination maximizes the number of droplets expelled by a single cartridge per pulse, while maintaining enough distance between nozzles to avoid midair droplet combination above the receiving solution.

To test for uniformity in droplet volume, droplets of blue ink were fired at 10, 100, and 1,000 Hertz for 600 seconds into a quartz cuvette containing 3 ml of distilled water. After calibrating a Hitachi U2000 UV/VIS Spectrophotometer to determine the relationship between absorbance and concentration of blue ink, a study of ejection volume reproducibility was performed. A pair of electrical contacts that fired a single droplet of blue ink upon the reception of a single pulse was wired to the driver electronics. The cartridge was then positioned over the mouth of the cuvette and a set of droplets was fired for 600 seconds at each frequency, measuring the absorbance of the solution in the cuvette every 120 seconds as measured by a stopwatch accurate to the nearest second. The curve of droplet volume versus time for the various frequencies was obtained. The most reproducible droplets were formed in the range of 100 Hz. It can also be shown that at 100 Hz, the standard deviation is only 0.66% of the average volume (17.72±0.12 picoliters).

Using the results of this experimentation, it was deduced that the forces acting at the pore, in the case of static equilibrium, are hydrostatic pressure and surface tension. Therefore, in order to replace the ink of the cartridge with another solution, hydrostatic pressure and surface tension must be equilibrated or the cartridge must be “primed.”

Hydrostatic pressure was mechanically controlled by filling the central chamber, which previously contained blue ink, with a polymer solution and sealed around the chamber using a sheet of silicone, cut to seal around the chamber, and a syringe. By adjusting the position of the syringe plunger relative to its barrel, a change in volume corresponded to a change in hydrostatic pressure. For solutions with low surface tension, increasing the volume of the chamber reduced hydrostatic pressure. Given high surface tension, decreasing the volume of the chamber increased the hydrostatic pressure.

However, serial droplet ejection effectively reduces to a pulsatile flow through the pores. This loss of volume causes the cartridge to lose its prime, often in minutes, when functioning for hours is desirable. As such, the pressurization system was removed and material properties of the polymer solution were modified to achieve a reasonable working-time (in the range of hours), without compromising integrity (mechanical and chemical stability) of the resultant hydrogel microstructures.

A working range of concentrations for droplet generation at atmospheric pressure was established to be between 0.1 wt. % and 1.25 wt. % of aqueous low viscosity sodium alginate. Beyond this point, cartridges were no longer outfitted with pressurization systems. Instead, HP51625A cartridges were opened using a Ryobi 9″ Band Saw equipped with a wood cutting blade, their contents were drained, and then the cartridge was washed repeatedly with distilled water to remove residual ink and prepare the cartridge lumen for refilling with a polymer solution. Once filled, a Gast Wet/Dry Vacuum Pump outfitted with Nalgene™ tubing was used to apply relative negative pressure to the foil containing the cartridge pores. After the polymer solution was pulled through the pores, laboratory tissue was used to remove any excess polymer solution from the face of the foil and the cartridge was ready to accept a signal from the reengineered cartridge driver.

b) Investigation of Aqueous Calcium Alginate Hydrogel Formation

Alginic acid sodium salt from Macrocystic pyrifera (Kelp) low and medium viscosities (algin), and calcium chloride dihydrate (>99%) were obtained from Sigma Aldrich. When algin is introduced into a calcium rich environment, the divalent calcium cation physically cross-links alginate molecules to form a hydrogel, such as those used in foods (e.g. ice cream, gummy candy) as a thickening agent and by physicians as a treatment for irritatable bowel syndrome.

Gel “strength” (characterized by material density, the extent to which the polymers physically cross-link, the charge densities of the constituents, and mechanochemical and thermal stabilities) affects the rate of release of a drug from each capsule.

Factors that affect gel strength are the electronegativity of the divalent cation, the guluronic to manuronic acid content ratio of the alginate used, and the concentration of the alginate solution. Calcium makes a strong biocompatible gel which degrades in the absence of ambient calcium. That is, calcium will diffuse from the microsphere to a calcium deficient environment, which is important for later modifications to the gel. Since alginate is a diblock copolymer composed of guluronic and manuronic monomeric units, and only guluronic acid contains a locus of negative charge (necessary for physical cross-linking with calcium), alginate rich in guluronic acid yields the strongest gel, both pre and post formation of the final coascervate product.

Factors that affect droplet shape beyond gelation determine the desired properties of the polymer solution. The distance the droplet travels from the pore to the receiving solution (drop height), the speed at which the receiving solution is stirred, the time it takes for the gel to cross-link sufficiently (which is directly related to the calcium concentration), and the difference in hydrophilicity between the polymer and receiving solutions all affect the shape of the resultant hydrogel.

Assuming that the receiving solution is aqueous calcium chloride, beyond some height, the resultant gel fails to penetrate the surface of the receiving solution. That is, drag forces cause the droplet to slow as it travels the distance between the nozzle and receiving solution surface. Below some impact velocity, the droplet fails to penetrate the receiving solution surface and “pancakes”. As drop height decreases, the tail region of the resultant teardrop shaped gel increases in size. At approximately 8.5 mm, using 1.0 wt. % medium viscosity algin and 1.0 wt. % aqueous calcium chloride, a nearly spherical gel is formed beyond impact.

A Corning Stirrer/Hot Plate with a regulated stir speed and seven variable settings was used to stir the receiving solution. When the stir speed was too high, the hydrogel would appear stretched or reproducibly ellipsoidal. When the stir speed was too low, the gel would form in a teardrop shape. At a stir speed setting of “2” (approximately 2 Hz), given the appropriate drop height, calcium concentration, and alginate concentration, the microspheres would take a nearly spherical form. However, variability in shape was quantitatively noticeable, and could likely be attributed to the irreproducibility of stir speed and the very large dependence of drop height upon the concentrations of the media.

Since stirring and drop height can limit reproducibility, experimental efforts were made to eliminate their roles in hydrogel formation.

c) Reformulation of the Receiving Solution

Alginate must reside in aqueous media in order for it to dissolve in any appreciable amount, and so in order to increase interfacial tension between the polymer and receiving solutions (negligible using two aqueous solutions), the hydrophilicity of the receiving solution had to be reduced. Reducing the hydrophilicity, or increasing the hydrophobicity, of the receiving solution causes the aqueous algin droplet to form a ball to minimize surface energy by minimizing interfacial surface area. By creating an environment in which the desired shape is most energetically favorable, the droplet has time to recover from impact before gelling (i.e., when the available calcium concentration is not exceedingly high) and the influence of drop height and stir speed as variables to droplet formation are eliminated.

The desired properties for the receiving solution were: calcium source availability, a high degree of biocompatibility, a highly hydrophobic nature, and miscibility with aqueous algin. Two types of calcium salts were considered, those with hydrophobic anionic components such as stearic acid calcium salt (Fluka) and oleic acid calcium salt (Sigma) and those with inorganic anions, such as calcium chloride dihydrate (Sigma-Aldrich). Calcium salts of organic molecules would provide a calcium source in a hydrophobic medium, however, their solubilities are prohibitively low and gellation would rely upon the diffusion of calcium across an interface.

To solubilize calcium chloride for the purpose of making a calcium rich receiving solution, alcohols and water were considered. Since aqueous solutions proved problematic in the previous experimentation, alcohols were investigated. Due to solubility, short hydrocarbon chain alcohols from methanol through pentanol were investigated. Ethanol was chosen as the salt carrier as it is miscible with water, dissolves up to 22 wt. % calcium chloride, and is the most biocompatible alcohol.

In order to increase the hydrophobicity of an ethanol salt solution, ethanol had to be mixed with a hydrophobic substance. After experimenting with various groups of organic compounds, alkanes were chosen to increase the hydrophobic nature of the ethanol, given their low viscosities relative to oils. n-Heptane (Sigma-Aldrich) in particular was chosen, as it is the most chemically inert alkane.

Given the reformulated receiving solution, gel shape depended only on calcium concentration, the proportion of ethanol (which acts as the calcium carrier) to n-heptane, and the frequency of droplet ejection. To make perfect spheres of calcium alginate gel reproducibly, calcium chloride concentration was determined to be optimal at 1.5 wt. % in a solution of one part n-heptane to one part ethanol. That is, the optimal concentration of calcium chloride solution allows sufficient time for the droplet to recover from its deformation upon entry and ball up, while causing gellation soon enough to avoid much deformation due to collision with its neighbors, as observed using light microscopy. The maximum frequency of firing (ejection of droplet from inkjet cartridge), while maximizing reproducibility was determined experimentally to be 250 Hz out of four nozzles per cartridge, for a total of 1 kHz production out of each cartridge, by visual inspection of the microspheres under high magnification.

### Example 2

### Cartridge Pressurization and Active Loading Schemes

At the pore of the Engineered Release Systems Capsule Generator (ERS CG) droplet generating device, interfacial tension and cohesive weak forces counteract hydrostatic pressure. Surfactants, ambient conditions, and solution density can be tuned to cause retention but allow expulsion of the media to be expelled. Presently, a dynamic pressurization is used to mechanically achieve the necessary equilibrium conditions at the pores of the ERS droplet generating device.

Many solutions have the appropriate ambient equilibrium conditions in the absence of pressurization. However, if the hydrostatic pressure greatly exceeds the interfacial tension and cohesive weak forces of the media to be expelled, vacuum can be applied by the pressurization system to achieve the desired equilibrium conditions. Otherwise if, the interfacial tension and cohesive weak forces greatly exceeds the hydrostatic of the media to be expelled, pressure can be applied to the media to be expelled to achieve the desired starting conditions.

Upon receipt of the square-wave pulse from the ERS CG driver electronics, the local pressure at the pore is transiently increased to expel individual droplets. By pressurizing the fluid reservoir of the ERS CG, the correct initial conditions can be met by a greater range of media which can be front-loaded for expulsion.

a) Methods and Materials

Pressurization is achieved by interfacing a linearly actuated piston/cylinder arrangement with the chamber containing media to be expelled by means of a polymer gasket. In one embodiment, a syringe is affixed to a machined piece of polycarbonate. After the ERS CG is filled with the media to be expelled, the silicone gasket is clamped between the machined polycarbonate and the fluid reservoir of the ERS CG. Once the seal has been made, the volume of the fluid reservoir and the volume of the piston/cylinder arrangement comprise the initial volume (Volₒ). By moving the piston relative to the cylinder, the plunger with respect to the barrel of the syringe, the total volume of fluid reservoir can be controlled (ΔVol). As explained by Boyle's Law: Pressₒ Volₒ=(Pressₒ+ΔPress)(Volₒ+ΔVol), varying the total volume of the fluid reservoir directly translates to controlling the pressure within the reservoir.

Specifically, high w/v % alginic acid and viscous active solutions often cannot be expelled from the ERS CG at atmospheric pressure. Additionally, surface active polymers in high concentrations (i.e., poly-ethylene glycol) reduce interfacial tension enough to disturb equilibrium conditions. However, with the application of positive pressure in the former and vacuum in the latter, front-loading conditions can be achieved allowing for more than one thousand droplets per second to be expelled from the ERS CG.

By expanding the range of the front-loadable actives, many more polymer blends can be used with the ERS CG. Specifically, pressurization is very important for working with solutions at the extremes of viscosity, density, interfacial tension, and weak cohesive forces. In addition to front-loading, these polymer blends can also be back-loaded with actives and other polymers as previously described.

### Example 3

### Polymer-Based Microstructures

To form a strong coacervate shell within the alginate template, a polycationic species, chitosan, is allowed to infuse into the calcium alginate template, replacing calcium as the cation source. The covalently bonded positively charged units of chitosan form a greater number of electrostatic interactions with any two given alginic acid chains giving rise to a capsule with a more robust, less permeable shell and a less robust, more permeable core.

a) Materials and Methods

As described in Example 1, calcium-alginate “templates” are generated by introduction of microdroplets, expelled from the head of a droplet generating cartridge, containing a low viscosity algin (alginic acid sodium salt) solution into a receiving bath containing a heptane/ethanol/CaCl₂ solution. Ethanol allows for the dissolution of a divalent cation and its complement (Ca⁺⁺ and chlorine ions) into a solvent miscible with a low viscosity, hydrophobic solvent (heptane). A multivalent-ion (calcium) source is required for physical cross-linking of the guluronic acid anionic centers between individual polymer chains. Heptane is responsible for increasing the interfacial tension between the droplets and the receiving solution, forcing each droplet to bead up into a sphere, avoiding generation of malformed microspheres (i.e. elipsoids, “teardrops,” “pancakes”, etc.). The ethanol acts as a calcium carrier and enters the droplet, causing it to swell temporarily, and promoting hydrogel formation.

In order to recover the templates from the heptane/ethanol/CaCl₂ solution, hydration by misting of the receiving solution surface is performed at a rate on the order of microliters of water per minute. Over time, the solution separates into a hydrophobic medium (heptane) and the hydrophilic medium (ethanol, salt, and polymer microspheres). Aqueous calcium chloride is then added at an increased rate to promote further hydration of the microspheres (diluting the ethanol in solution). Finally, the mixture is spun in a rotary evaporator to purge ethanol from the system. Heptane may then be drawn off, or made to evaporate by further heating in the rotary evaporator with added water, as water will be drawn off with the heptane as water and heptane have similar boiling points at atmospheric pressure −100 degrees C. and 98.5 degrees C., respectively. Addition of water is also necessary to avoid overly concentrating the aqueous salt solution remaining, containing the polymer microspheres.

Once the templates are formed, dextran (a charge-neutral polysaccharide) is added to the suspension to match the density of the surrounding solution to that of the microspheres. This precludes collection of the microspheres at the bottom of the mixing container due to separation by weight. Next, a low-viscosity chitosan solution, approximately 0.5 wt. %, is prepared. The microspheres are agitated (by stirring) in a mixing chamber and added to the volume of chitosan solution by way of a HPLC pump. The pump is to be programmed to vary the rate of pumping to introduce microspheres at various times (i.e., pump one microsphere during second 37, no microspheres during second 38, and ten in sequence during second 39) given the concentration of microspheres in the mixing source. The time that the microspheres are allowed to sit in the chitosan solution determining the “wall thickness” of the capsule, of how much chitosan is to enter the template, and how great the radial penetration is for some given critical concentration of chitosan. A steep concentration gradient is to be achieved along the radius of the microsphere (forming a “capsule” with a gel core). As the doping concentration of chitosan increases per capsule, so does the independence to effusion of a drug from the capsule. The permeability of the outer chitosan-rich region can be orders of magnitude smaller than that of the inner alginate core, as is the case with conventional macroscopic gel capsules where only the outer capsule is a barrier to diffusion, and is not be dissolved during release, and varies from capsule to capsule (or set of capsules to set of capsules) allowing for the engineering of drug-specific, novel release schemes. The capsules are then centrifuged and the chitosan supernatant solution decanted and the capsules resuspended in whatever final media is most preferable.

b) Discussion

From a drug stability standpoint, as far as introducing the drug into capsule is concerned, the optimal time to load the capsule is at the end of capsule formation. Adding a concentrated solution of the drug to the microspheres directly after the chitosan solution is decanted allows for resuspension of the capsules in a drug rich environment. Drug infusion will proceed over time, and may be stopped by again centrifuging and decanting the supernatant fluid to recover the remaining drug. Quantity of drug in the dose, or population of capsules, can be calculated by quantifying the concentration of the drug in the added volume before and after capsule infusion. Experimentally, both the time necessary for dilution per type of drug and percent lost (this quantity may be recovered by centrifuging again and decanting) can be quantified by analytical means (e.g., HPLC for insulin, GC for Heparin, etc.)

Depending upon the sort of drug to be encapsulated, the core may be left as a gel at the time of drug addition or dissolved by adding a concentrated sodium citrate solution to force displacement of the calcium ions in the gel by sodium, “resolubilizing” the alginate over a short period of time. The capsules can be stored dry (by lyophilization) or in fluid media. If stored in fluid, a hydrophobic medium is preferred for water soluble molecules, and a hydrophilic solution is preferred for oleophilic compounds, in order to prevent premature effusion.

### Example 4

### Polymer-Based Microcapsules

Although microstructures can be made into microcapsules by liquefying the core of the microsphere, in some instances it is of interest to form microcapsules without using a template. Towards this end, a protocol was designed for producing microcapsules in a single step. Specifically, to make a calcium alginate microcapsule, inkjet cartridges are used to expel a calcium chloride solution into a receiving algin solution.

By introducing the calcium into a receiving polymer solution, a layer of calcium alginate hydrogel is formed around the calcium chloride droplet. This results in a constant volume liquid core, while allowing for controlled variation of the shell thickness through varying concentrations of the polymer solution and cross-linking agents. Contrarily, capsules formed by liquefying the cores of template microspheres yield capsules of constant total volume, while allowing for controlled variation of the shell thickness. Additionally, in comparison with the capsules formed from microsphere templates, the microcapsules have a decreased potential mechanical stability inherent in a liquid-core design.

a) Methods and Materials

To produce liquid-core calcium alginate microcapsules of a single wall thickness, a calcium rich solution is expelled from the head of a modified HP 51625A inkjet cartridge into a receiving bath containing sodium alginate solution. Application permitting, the calcium rich solution is composed of a combination of heptane (a hydrophobic solvent) and ethanol, in which calcium chloride is dissolved. The heptane ethanol solution is desirable for creating a large interfacial tension between the two solutions to ensure the spherical shape of the capsule walls.

For the purpose of encapsulating cells (see Example 9), a combination of 0.5 wt. % calcium chloride, dextran (a thickening agent, used to promote penetration into the receiving solution) and sucrose (an inert molecule) is adjusted to physiological osmolarity (300 m0sm), with varying amounts of dextran and sucrose, depending upon the cell type. The experiments are conducted inside a sterile incubator at body temperature to promote long-term cell viability.

Since the capsule wall-thickness depends principally upon the amount of available calcium in the liquid core and the concentration of the sodium alginate in the polymer receiving solution, wall thickness can be varied during capsule production by diluting, or concentrating either solution during capsule production. Furthermore, the degree to which the variation among wall-thicknesses of the capsules is continuous can be controlled based upon the frequency of droplet ejection, and the flow rate at which the diluent is added to either the solution in the inkjet cartridge or the receiving bath.

As in the case of forming solid alginate microspheres, the microcapsules of varying diameter can also be used as templates. To increase mechanical strength, the calcium alginate capsules can be transferred to a polyanionic solution (e.g., chitosan) to form a polymer blend. Additionally, components of a chemically cross-linked polymer can be added throughout the walls of the capsules to control material properties or to induce functionality for site-specific delivery.

b) Discussion

Microcapsules can be used to control the release of therapeutic agents by means of profile approximation by sigmoidal summation in a similar manner to the capsules formed from solid microsphere templates. However, to predict the release profile, the variability in total capsule volume must be accounted for.

### Example 5

### Characterization of Chitosan Wall Thickness

a) Characterization by Analytical Means

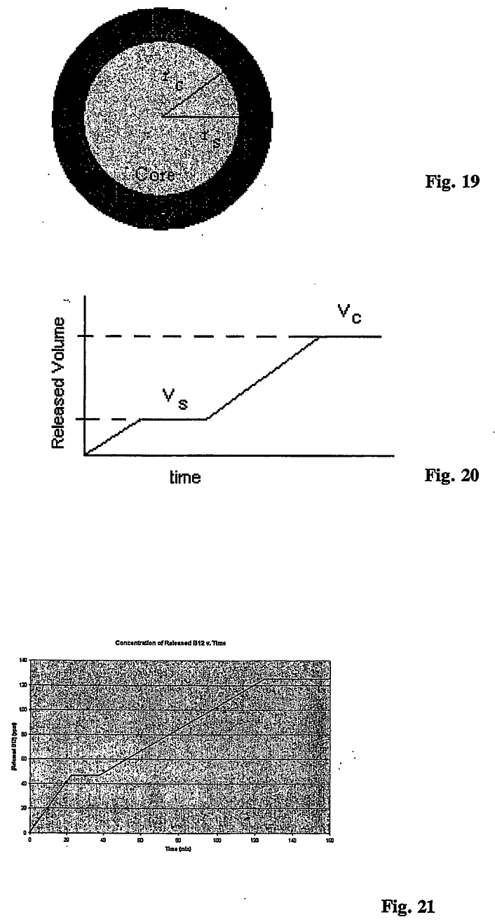

The “wall thickness” of the chitosan coating is responsible for the difference in time to maximum release from capsule to capsule (FIG. 19). Since there is analyte present in both the outer wall and in the core of the capsule, the release profile per microsphere, unless the outer wall is purged of its contents, contains two plateaus corresponding to the two release maxima (i.e., the time to maximum release of the outer wall contents, which is seen first, and time to release of the core contents, which marks the global “time to release”). The penetration depth (or thickness) is quantified by taking the ratios of the plateau heights per microsphere batch.

Assuming an even distribution of analyte throughout the microsphere, rₛ is measured using digital imaging techniques. The release profile of the polymer blend microcapsule takes the form of the curve as seen in FIG. 20. That is, the absolute thickness of the wall is not of concern, but rather the relative thickness from microsphere to microsphere, given the range between some maximum and minimum chitosan contents, as well as the corresponding times to maximum release.

$${r}_{c} = {( {r}_{s}^{3} - ( \frac{{r}_{s}^{3} {V}_{1}}{{V}_{2}} ) )}^{1 / 3}$$

$${t}_{s} = {r}_{s} - {r}_{c}$$

V₁ and V₂ correspond to the volume of the active released from the shell and core, the level of the first and second plateaus of the sigmoidal release curve respectively. The above equation is used to delineate the core and shell volumes of solid-core microstructures, of which the rₛ and release profile (corresponding to V₁ and V₂) are known, and the r_{c} is to be determined. Since there is no way to visually distinguish between the core and the shell in some of our solid-core formulations, the equation gives a means of characterizing shell thickness based upon the release curve that allows for a comparison within a single and among populations with different effective shell thicknesses.

b) Characterization by Microscopy

Absolute wall thickness can be characterized as follows: Chitin, the form of chitosan preceding deacylation, tagged with a visible dye (e.g., “chitin azure”), or a fluorescent dye (e.g., FITC labeled chitin) is added to the chitosan bath in the step prior to the introduction of the template microspheres by way of a HPLC pump. The tagged chitin enters with the rest of the chitosan and takes residence within the microsphere during capsule formation. The microsphere is then inspected by confocal microscopy, yielding planar scans of the radial dye distribution across the depth of the microsphere (that is, X-Y scans across the Z-direction) using the appropriate light source and filters to distinguish the shell from the core.

### Example 6

### Review of Potential Drug Targets and Methods of Release Profile Analysis

In order to characterize the in vitro release of selected analytes from their corresponding sets of engineered capsules, and to satisfy preliminary testing for determination of eligibility to conduct FDA regulated clinical trials, FDA dissolution apparatus type II and appropriate USP dissolution methods, as well as USP-prescribed dissolution media (e.g., simulated gastric and intestinal media, simulated blood plasma and tissue fluids) are employed. The following methods and prescribed release patterns are useful insofar as each helps to determine a trend in vivo. Clinical work must follow to establish efficacy in the body. Some compensation for clinical findings will follow in the engineering of the release systems, not to be seen in the laboratory environment.

During development of the system, model analytes (those with special characteristics (e.g., visible dyes, those with easily targeted, characteristic chromophores) were used for testing using a Hitatchi U2000 UVNIS Spectrophotometer as well as an inverted microscope and a real-time image capturing system (Olympus CKX-41 Inverted Light Microscope with Phase Relief and Hoffman Relief Phase systems, as well as an Olympus DP-12 CCD Microscope Camera).

FIG. 21 provides a representative result of Vitamin B 12 release over 2.5 hours from Alginate-(Poly-L-Lysine) capsules of uniform wall thickness. The result is a curve characteristic of capsules with analyte present in (i.e., not purged from) the outer wall, which is useful for determining relative wall thickness in populations of microspheres.

### Example 7

### Investigation of Microcapsule Dimensional Response

It has been observed that microcapsules can be forced to contract significantly by drastically changing the ambient salt concentration and water content. Via a heretofore unseen solvent-exchange mechanism, it has been observed that volumetric contraction greater than an order of magnitude can be achieved by decreasing salt concentration and increasing water content drastically. Models and experimentation by Solis demonstrate that up to a 30% volumetric change can be achieved by taking advantage of the property of lower critical solution temperature (LCST), an energetic phenomenon exhibited by a sub-set of polymers.

We observe significant volumetric change without varying ambient temperature, typically undesirable when working with bioactives. It is proposed that the mean-free-path of the individual polymer chain average length, and therefore average volume occupied, is dependent upon electrostatic and hydrophobic forces, analogous to protein folding. Both proteins and physically cross-linked polymers can be modeled as long chains of charged or neutral sub-units. Based upon the ambient conditions, the hydrophobic and hydrophilic regions rearrange to form a lowest energy conformation. In the observed case, alginic acid composed of repeating subunits of mannuronic and guluronic. In a highly ionic, slightly non-polar ambient solution, mean free path of alginic acid is significantly greater than in distilled water.

Osmotic contraction via a solvent-exchange mechanism, whereby a less polar, more highly ionic strength solvent is exchanged for a more polar, weaker ionic strength solvent can be used to control the physical parameter of volume. Additionally, contraction can be used to effectively concentrate actives, where by actives is defined as any front-loaded molecular species.

a) Materials and Methods

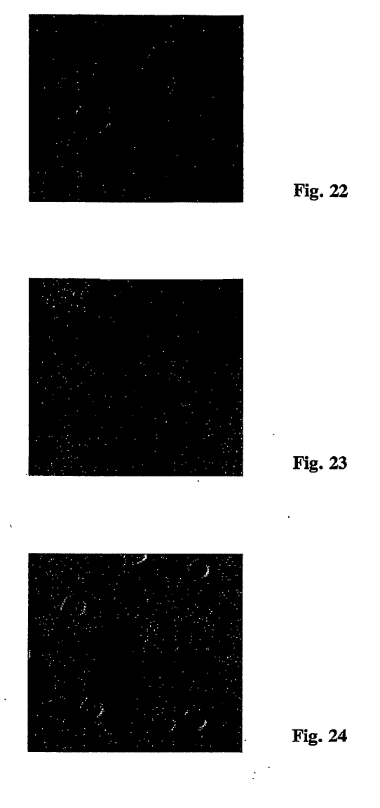

Using an ERS CG, aqueous droplets of 0.67 w/v % low viscosity sodium alginate are expelled into a receiving solution of 0.25 w/v % calcium chloride in ethanol receiving solution. When the ambient receiving solution is exchanged for double-distilled water, the resultant population of microcapsules experiences an order of magnitude reduction of volume (FIG. 22).

It is worthy of note that in this particular incarnation, no secondary solvent is necessary to achieve nearly perfectly spherical alginate microcapsules. In the above experimental methods, the transient interfacial tension created between the aqueous polymer solution (i.e., 0.67 w/v % sodium alginate) and the primary solvent (i.e., 0.25 w/v % calcium chloride in ethanol) provides enough energy to the polymer solution to obtain the spherical conformation. This is of particular use when encapsulating actives that are sensitive to the composition of the ambient environment, but for which nearly perfectly spherical microcapsules are desired.

In FIG. 23, imaged directly after the ambient solution was changed, the smaller spheres surrounding the microcapsules are believed to be composed of solution within the microcapsules that is then exchanged with the ambient. We postulate that the resultant environment within the microcapsules is a complex mixture of hydrophilic and slightly hydrophobic regions at low ionic strength, thereby facilitating the osmotic contraction seen in FIG. 24.

### Example 8

### Environmentally Dependent Delivery of Ultra-High Molecular Weight Active

Calcium alginate microstructures can be either front- or back-loaded with a single, or multiple environmentally-cued polymer solutions based upon the size of the monomer in relation to the molecular mass cut-off of the template. Environmentally-cued is defined as a polymer solution that is subject to change in the form of gelation or cross-linking upon changes in the ambient solution (i.e., pH dependent gelation of methacrylic copolymers). Once the environmentally-cued polymer occupies the alginate template, gelation or cross-linking is cued by changing the ambient condition. Afterwards, the alginate template is dissolved with a monovalent salt of a calcium chelating agent (i.e., sodium citrate) leaving the environmentally dependent polymer in the geometry of the alginate template in either a fluid-core or solid-core geometry. Environmentally-cued microstructures are of particular interest for delivery of molecules higher than the molecular mass cut-off of the environmentally sensitive/scaffold polymer blend in an environmentally dependent and/or site-specific fashion.

Given the in vivo conditions of the gastrointestinal and circulatory systems, environmentally-cued release can be tailored to target specific delivery environments. For example, methacrylic copolymers are insoluble in acidic aqueous media and soluble when exposed to aqueous media with a pH slightly below that seen in the small intestines. Oral administration of pH dependent microstructures effectively provides enteric coating for the active agents due to the pH dependence of solubility. In the gastric environment, the active is protected from strongly acidic conditions by the shell of the fluid-core capsules. When the environmentally dependent microstructures reach the jejunum, ileum, and duodenum, they are exposed to a higher pH causing dissolution of the shell. In the nearly neutral environment, the contents of the particles will be released at the point of the greatest nutrient uptake in the body.

Using a novel manufacturing process, ERS has succeeded in producing individual pH sensitive microcapsules (both fluid-core and solid-core) each containing picoliters of an aqueous solution.

At approximately neutral pH, the polymer shell becomes soluble, making the pH sensitive fluid-core microcapsules a viable candidate for enteric-protective encapsulation. Bioactive macromolecules are to be encapsulated in the pH sensitive microcapsules in order to ensure their safe passage through the gastric components of the digestive tract, into the intestinal system. Preferential adhesion of microcapsules to the intestinal lining is to precede release when necessary.

The Engineered Release Systems Capsule Generator was used to produce the templates for as many as one thousand pH sensitive microcapsules each second. The driver electronics currently employed for research-scale operation can drive ten, individual ERS CGs in parallel.

Additionally, solid-core, environmentally sensitive microcapsules can be used to extend, sustain, delay, and control the pharmacokinetics of the encapsulated active as well. Moreover, solid-core environmentally sensitive microcapsules allow for the environmentally sensitive coating of any ERS solid-core formulations. Since the specific formulation of the environmentally-dependent polymer shell, template guided formation of microspheres extends the range of capabilities to targeting environments based upon environmental cues, as well as to allowing the use of external factors to cue release.

### Example 9

### Single-Cell Encapsulation

Since the advent of tissue engineering, researchers have worked towards devising schemes for encapsulating cells in cell culture media (i.e., agar, alginate, cellulose derivatives, etc.). The encapsulating polymer blends are to protect cells from immune response.

Additionally, polymer membranes around cells provide opportunity for site specific binding of the polymer-cell systems in vivo without modifying the cells themselves. Similarly, providing cells the ability to preferentially bind to regions of tissue constructs can be of great value to the field of artificial tissue engineering. Genetically modified, encapsulated cells are ideal for delivery of spatially dependent growth hormones in artificial tissue constructs.

Less attention has been paid to the possibility of cell encapsulation for drug delivery within the human body. Encapsulation of β-pancreatic islet cells, which naturally produce insulin, have received the most attention as a potential means of delivering insulin for extended periods of time in diabetic patients in the future. It is likely, however that any sort of cell expressing a protein via recombinant DNA technology could be encapsulated, protected from an immune response, and implanted in the body so that it can make and release its metabolic products throughout the lifetime of the cell.

However, this same barrier (the encapsulating medium) that affords the cell protection, also serves as a barrier to receiving metabolites and excreting waste products. For this reason, the greatest surface area to volume ratio per cell is desired. In other words, the most advantageous sort of cell encapsulation, in terms of extending the life time of the cell and maintaining cell phenotype, is single-cell encapsulation.

a) A Review of Polymer and Receiving Solutions Suitable for Cell Encapsulation and Culture

All four categories of polymer and receiving solutions combinations discussed for drug delivery (single and dual component physically and chemically cross-linked polymer systems) are applicable to cell encapsulation. However, due to the high sensitivity of living cells to their surrounding environment, harsh cross-linking agents such as ultra-violet light and nocuous chemicals such as divinyl sulfone cannot be used. In general, physical cross-linking processes tend to be less detrimental to cell viability than chemical cross-linking processes. So, physically cross-linked polymers are considered, even though electrostatic, intermolecular bonds are weaker than covalent bonds. Specifically, agar and calcium alginate encapsulation media protocols are employed.

Agar, perhaps the most common solid cell culture media, comes in a powdered form. Dependant upon the type of cell being cultured, additives such as lyophilized sheep's blood or minimal media can be added. Additionally, drugs like Kanamycin can be added to agar solutions, which can inhibit bacterial growth around the encapsulated cells, thereby increasing the storage potential of encapsulated cell suspensions. Once powdered agar is mixed in the appropriate proportions with water, it is heated in order to allow the powder to become fully soluble. At body temperature, agar solution takes on the order of minutes to solidify into a gel. When preparing the warm agar to be loaded into the inkjet cartridges, physiological pH, temperature, and solution osmolarity can be monitored and maintained. Physiological pH can be achieved through buffering, temperature can be regulated by the addition of Peltier heater/coolers to the body of the inkjet cartridge, and osmolarity maintained by the adjusting of concentrations using sucrose (or any simple sugar) as an inert substitute for bodily electrolytes (which are known to interfere with the gelling process) when matching the osmolarity of interstitial fluids, without increasing viscosity tremendously as is needed.

Calcium alginate, is also a good candidate for cell encapsulation because the concentration thresholds necessary for gelation are well below 300 mOsm and the critical concentration at which calcium becomes toxic to most cell types. In addition, an elevated temperature (body temperature) aids in keeping the viscosity of the sodium alginate low, while the gelation process of electrostatic interaction has been shown to have little or no effect on cell viability (relative to cells grown on conventional, uncharged media such as agarose gel).

Paralleling the protocols for microsphere and capsule formation, cell coatings can be accomplished by loading inkjet cartridges with an aqueous sodium alginate cell suspension as the expellant from the capsule generator. Cell encapsulation can be accomplished by loading inkjet cartridges with an aqueous calcium chloride cell suspension as the expellant from the capsule generator. Both the polymer and receiving solutions can be pH, osmolarity, and temperature matched in order to maintain the greatest cell viability. The pH matching can be achieved through buffering, while temperature can be maintained by Peltier heater/coolers and osmolarity matched using a simple sugar or other inert molecules. However, neither agar, nor calcium alginate can protect the encapsulated cells from an immune response if injected. Therefore, these initial methods of cell encapsulation serve as templates. The templates can be displaced by more biocompatible, potentially functionalizable polymers such as cellulose derivatives. Once the cells have been encapsulated, the encapsulation matrix provides an optical barrier and permeability barrier to the external environment for the cells allowing for a greater range of allowable chemical reactions. Afterwards chelating agents and/or substrate specific enzymes such as sodium citrate, agarase, etc., can be used to liquefy the remaining template material.

Another major concern surrounding the introduction of encapsulated cells into the blood stream is their effects on the liver and kidneys. The encapsulating matrix can either dissolve or can be removed (by some internal immune process such as macrophage phagocytosis) before encountering these organs, or the encapsulated cells can be confined to a region of the body such that they are not allowed to freely circulate.

b) Testing and Experimental Protocols for Ejecting Cells from the HP 51625A Inkjet Cartridge

Single-cell encapsulation has been achieved using HP 51625A ink jet cartridges and the processes above. However, the use of piezoelectrically actuated cartridges for droplet expulsion (such as those produced by Epson) can potentially lead to a higher expected yield of viable cells by avoiding any heating and heat-induced cytolysis during thermally induced droplet ejection. Aside from the mechanism for droplet ejection, the nozzle size of the cartridge is important for determining what cells can be encapsulated. Inkjet cartridges of varying nozzle sizes from 5 to 50 microns in diameter are readily available, and the HP 51625A cartridge has 30 micron diameter nozzles.

The nozzle diameter must exceed cell diameter, but not twice the cell diameter. Therefore, HP 51625A cartridges can accommodate cells ranging in diameter from approximately 16 to 29 microns, which includes insulin producing bovine and porcine β-pancreatic islet cells. To prepare cells for ejection from the cartridge, the cells are first cultured. Trypsin is then added to the cell culture media for some time during which peptide bonds causing cellular adhesion to the growth substrate can be broken. Cells are then filtered to remove any remaining clumps through a filter with a pore diameter equal to that of the nozzle diameter. After the filtered cells are pelleted by a centrifuge, the supernatant fluid is decanted and the cells are resuspended in Dulbecco's Modified Eagle's Medium (DMEM) to deactivate trypsin. Having been resuspended in DMEM, the cells are spun down again using a centrifuge and are then resuspended in a 300 mOsm sodium alginate/sucrose solution. The sodium alginate portion of the solution has been experimentally determined to be 0.67 wt. % and the solution adjusted to 300 mOsm (with sucrose) and physiological pH (by buffering). To coat cells in agar, the cells must be resuspended in a 300 mOsm agar/sugar solution (rather than in sodium alginate/sugar).

Cells are introduced into the agar/sugar solution after the agar/sugar solution is first heated to fully dissolve the powder and then cooled to 37 degrees C. in a water bath. The cell suspension is then loaded into the inkjet cartridge. In order to avoid aggregation, which can clog the nozzle of the cartridge, a rice-grain sized stir bar is placed at the bottom of the chamber containing the cell suspension. Finally, the cartridge is primed using a vacuum pump and a syringe is used to equilibrate pressure after priming to ensure the reproducibility of droplet formation for the necessary time period. A schematic diagram of the inkjet cartridge setup containing the cell suspension positioned over the receiving bath is shown, see note on FIG. 25.

c) Testing and Experimental Protocols for Encapsulating/Coating Single Cells

Once the inkjet cartridge has been loaded with the cells suspended in the polymer solution they are fired at low frequency into the appropriate receiving solution. The cells suspended in sodium alginate are fired into a 300 mOsm calcium chloride/sugar solution at physiological pH and temperature sitting on a stirring/hot plate. Variables such as height, stirring rate, and frequency can be adjusted to get the desired shape of the coating.

After the calcium alginate has had time to gel, the receiving solution is diluted 2:1 with an isoosmolar aqueous sugar solution so that chitosan can be added. Aqueous chitosan, solubilized using the Brookfield method, is then added to the receiving bath to form a coacervate shell. Finally, the cells are pelleted using a centrifuge and then resuspended in sodium citrate to dissolve the remaining calcium alginate, leaving the desired chitosan alginate coating or shell. All solutions added to the receiving bath can be at physiological temperature, pH, and osmolarity in order to ensure the greatest cell viability. For cartridges containing a cell suspension in aqueous agar/sugar, the droplets are ejected into a 37 degree bath of a hydrophobic, biocompatible solution (i.e., light mineral oil).

After the agar has had a chance to gel, an aqueous cellulose derivative solution at the appropriate physiological conditions. When the aqueous cellulose solution is added, a separation between the hydrophilic and hydrophobic layers occurs and the encapsulated cells reside in the aqueous solution allowing cellulose derivative to diffuse into the agar coating. To chemically cross-link the cellulose, the appropriate cross-linking agent is provided (i.e., UV light, DVS, etc.). Finally, agarase is added to liquefy the agar gel, leaving cells coated and encapsulated in cellulose. At the end of either protocol, the cells are prepared for separation of microspheres containing live cells from all other microspheres.

The encapsulated cells are pelleted by centrifugation, and resuspended in an aqueous solution of DMEM and calcein-AM (one component of a conventional “live/dead stain”). Live cells produce various non-specific esterases which cleave calcein from the protecting group allowing it to fluoresce at a wavelength of 517 nm when excited by light of a wavelength of 494 nm. The staining only occurs locally within the living cell so that the living encapsulated cells can be separated using flow cytometry techniques.

d) Discussion

Single-cell encapsulation allows for the greatest possible duration of cell viability (or cell storage) by maximizing the surface area to volume ratio available for each cell to obtain nutrients and excrete waste products. Additionally, as opposed to other cell encapsulation techniques, single-cell encapsulation allows for the management of individual cells by their individual polymer membranes. Additionally, cell encapsulation is preferable to cell coating when the encapsulated cells can undergo growth. To encapsulate a single cell using an inkjet cartridge (or any MEMS device), a few engineering and biological principles must pervade all steps of the process. The encapsulating device must produce capsules of a volume greater than that of the cell, but not greater than twice the volume of the cell in order to ensure that no more than one cell is expelled per pulse breadth. Biological conditions favorable to cellular metabolism (i.e., temperature, physiological osmolarity, etc.) should be maintained throughout the protocol.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A population of microstructures comprising a permeable polymer shell, wherein the standard variance in the volume of the microstructures is less than or equal to 10% of the mean, and wherein the diffusion characteristics of the polymer shell vary within the population of microstructures.

2. The population of microstructures of claim 1, wherein the diffusion characteristics vary as a result of variable thickness of the shells of different microstructures in the population.

3. The population of microstructures of claim 2, wherein the thickness of the shells varies continuously in the population.

4. The population of microstructures of claim 1, wherein the variance of the diffusion characteristics of individual microstructures provides for a defined release profile of an active agent encased in the microstructure.

5. The population of microstructures of claim 4, wherein the release profile is a sigmoidal summation profile.

6. The population of microstructures of claim 1, wherein the microstructures are spherical.

7. The population of microstructures of claim 1, wherein the mean diameter of the microstructures ranges from about 1 micron to about 100 microns.

8. The population of microstructures of claim 3, wherein the mean diameter of the microstructures ranges from about 5 microns to about 50 microns.

9. The population of microstructures of claim 1, wherein the shell is a cationic cellulose derivative in an admixture with an anionic block copolymer.

10. The population of microstructures of claim 9, wherein the cellulose derivative is chitosan and the block copolymer is alginate.

11. A population of microstructures having a volume of less than or equal to about 10 nL comprising a cross-linked polymer, wherein the standard variance in the volume of the microstructures is less than or equal to 10% of the mean.

12. The population of microstructures of claim 11, wherein the microstructures are spherical.

13. The population of microstructures of claim 12, wherein the mean diameter of the microstructures ranges from about 1 micron to about 100 microns.

14. The population of microstructures of claim 13, wherein the mean diameter of the microstructures ranges from about 5 microns to about 50 microns.

15. The population of microstructures of claim 11, wherein the polymer is alginate cross-linked with calcium.

16. The population of microstructures of claim 11, further comprising a permeable polymer shell.

17. The population of microstructures of claim 16, wherein the shell is chitosan-alginate.

18. The population of microstructures of claim 17, wherein a thickness of the coating on each microstructure particle varies from other particles.

19. The population of microstructures of claim 17, wherein a thickness of the coating on each microstructure particle is substantially identical.

20. The population of microstructures of claim 11, further comprising a cell embedded in the cross-linked polymer.

21. A population of microstructures having a volume of less than or equal to about 10 nL comprising a permeable polymer shell, wherein the standard variance in the volume of the microstructures is less than or equal to 10% of the mean.

22. The population of microstructures of claim 11, further comprising a cell inside the microstructure.

23. The population of microstructures of claim 11, further comprising an active agent inside the microstructure.

24. A method of forming a population of microstructures, which method comprises introducing drops of a polymer solution into a receiving solution under conditions that permit cross-linking of the polymer in the receiving solution, wherein the drops have a standard variance in the volume that is less than or equal to 10% of the mean.

25. The method of claim 24, wherein the polymer solution is an aqueous solution and the receiving solution contains a hydrophobic component.

26. The method of claim 25, wherein the polymer solution is an alginate solution, and the organic solution is a mixture of a hydrocarbon and an alcohol comprising a calcium salt in a concentration sufficient to cross-link the alginate.

27. The method of claim 24, wherein the polymer solution is an aqueous solution comprising cells, wherein each drop comprises on average a single cell, and the receiving solution is also an aqueous solution.

28. The method of claim 27, wherein the polymer solution is an alginate solution, and the receiving solution comprises a calcium salt in a concentration sufficient to cross-link the alginate.

29. The method of claim 24, further comprising contacting the microstructure with a polymer, wherein the polymer interacts with and stabilizes the cross-linked polymer.

30. The method of claim 29, wherein the polymer is chitosan.

31. The method of claim 29, further comprising dissolving the cross-linked polymer cross-links.

32. The method of claim 31, wherein the polymer is alginate cross-linked with calcium contacted with a calcium chelating agent.

33. The method of claim 32, wherein the calcium chelating agent is sodium citrate.

34. The method of claim 27, further comprising contacting the microstructure with a permeable polymer, wherein the polymer interacts with and stabilizes the cross-linked polymer.

35. The method of claim 34, wherein the polymer is chitosan.

36. The method of claim 34, further comprising dissolving the polymer cross-links.

37. The method of claim 36, wherein the polymer is alginate cross-linked with calcium contacted with a calcium chelating agent.

38. The method of claim 24, wherein the drops are formed in a drop-forming apparatus comprising an orifice, a polymer solution supply reservoir, an activation element, and a controller.

39. The method of claim 38, wherein the apparatus is a modified inkjet printer cartridge.

40. The method of claim 38, wherein the apparatus employs inkjet printer cartridge components modified for forming polymer solution drops.

41. The method of claim 29, further comprising loading the microstructure with an active ingredient.

42. The method of claim 41, wherein the loading comprises gradient diffusion.

43. A drop-forming apparatus comprising a plurality of orifices of uniform size spaced far enough apart so that drops ejected from the orifices do not combine, a reservoir in liquid communication with the plurality of orifices, and an activation means for ejecting drops from each orifice.

44. The apparatus of claim 43 wherein the orifices are formed in metal foil.

45. The apparatus of claim 44, wherein the metal foil is gold foil.

46. The apparatus of claim 43, wherein each orifice has a diameter of about 30 microns.

47. The apparatus of claim 43, wherein the distance between each orifice is an order of magnitude greater than the diameter of each orifice.

48. The apparatus of claim 43, wherein the activation means comprises a controller and an activation element.

49. The apparatus of claim 48, wherein controller is an amplified constant pulse generator and the activation element is a resistor.

50. A population of microstructures comprising alginate cross-linked with calcium, wherein the standard variance in the volume of the microstructures is less than or equal to 5% of the mean.

51. The population of microstructures of claim 50, further comprising a chitosan-alginate shell.

52. The population of microstructures of claim 51, wherein a thickness of the chitosan shell on each microstructure particle varies from other particles.

53. The population of microstructures of claim 52, wherein the thickness of the chitosan shell varies continuously in the population.

54. The population of microstructures of claim 52, wherein the variance of the shell thickness of individual microstructures provides for a defined release profile of an active agent encased in the microstructure.

55. The population of microstructures of claim 54, wherein the release profile is a sigmoidal summation profile.

56. The population of microstructures of claim 51, wherein a thickness of the chitosan coating on each microstructure particle is substantially identical.

57. The population of microstructures of claim 51, further comprising an active agent.

58. The population of microstructures of claim 50, further comprising a cell in the alginate.

59. A population of microstructures comprising a permeable chitosan shell, wherein the standard variance in the volume of the microstructures is less than or equal to 10% of the mean.

60. The population of microstructures of claim 59, further comprising a cell inside the microstructure.

61. The population of microstructures of claim 59, further comprising an active agent inside the microstructure.
